(19) United States
(12) Patent Application Publication
(10) Pub. No.: US 2026/0188450 A1
(43) Pub. Date: Jul. 2, 2026

(54) SYSTEM AND/OR METHOD FOR PROCESSING OF ELECTRONIC TRANSACTIONS FOR HEALTHCARE DATA

(71) Applicant: Lunar Systems Inc., San Francisco, CA (US)

(72) Inventors: Gautam Sivakumar, San Francisco, CA (US); Dustin C. Chang, San Francisco, CA (US); Indu T. Nedumpara, San Francisco, CA (US); Jonathan P. Luo, San Francisco, CA (US)

(21) Appl. No.: 19/431,918

(22) Filed: Dec. 23, 2025

Related U.S. Application Data
(60) Provisional application No. 63/739,335, filed on Dec. 27, 2024.
(60) Provisional application No. 63/739,332, filed on Dec. 27, 2024.
(60) Provisional application No. 63/739,326, filed on Dec. 27, 2024.

(51) Int. Cl. G16H 15/00; G06F 9/54

(52) U.S. Cl. CPC G16H 15/00; G06F 9/542

(57) ABSTRACT

Systems, methods, and computer-readable media are disclosed for processing electronic transactions. In some embodiments, a server computing device receives a plurality of event objects related to a patient’s encounter with a health care system. Semantic extraction and vector embedding on parameters from the event objects to update a state of a patient graph, which maintains a unified record of the patient.

### CROSS REFERENCE TO RELATED APPLICATIONS

This patent application claims the benefit of priority to U.S. Provisional Patent Application No. 63/739,326, titled UNIFIED MANAGEMENT IN HEALTHCARE SYSTEMS, filed on December 27, 2024; U.S. Provisional Patent Application No. 63/739,332, titled UNIFIED MANAGEMENT IN HEALTHCARE SYSTEMS, filed on December 27, 2024; and U.S. Provisional Patent Application No. 63/739,335, titled UNIFIED MANAGEMENT IN HEALTHCARE SYSTEMS, filed on December 27, 2024. The disclosures of each of the foregoing applications are hereby incorporated by reference in their entireties.

### BACKGROUND

### 1. Field

This disclosure relates to methods and/or techniques for processing electronic transactions affecting electronic health data.

### 2. Information

The processing of healthcare transactions, from the moment a patient has an encounter with a service provider to a final financial settlement, is a notoriously complex and inefficient process. Conventional approaches typically rely on a fragmented ecosystem of disparate information systems that are not designed to communicate seamlessly with one another. For example, a clinician documents a patient encounter in an Electronic Health Record (EHR) system, which is primarily designed for clinical data management. Separately, administrative staff must manually review these clinical notes, abstract relevant procedures and diagnoses, and translate them into billing codes for entry into a distinct practice management or billing system.

This manual translation and re-entry of data between disconnected clinical and financial systems is a significant source of error, inefficiency, and delay. Data inconsistencies frequently arise, leading to claim denials that require further manual intervention, appeals, and resubmission. The entire process involves multiple handoffs—from the provider to a claims clearinghouse, then to a third-party payer's adjudication system, and back again—with each step introducing potential delays and points of failure. This fragmentation results in protracted revenue cycles, a high administrative burden on provider staff, and significant revenue leakage due to missed charges or un-reconciled payments.

Furthermore, this conventional model lacks transparency for all stakeholders. Providers, payers, and patients often have different views of the status of a transaction at any given time, making it difficult to track progress or understand financial responsibilities in a timely manner. The lack of a unified, auditable, and trusted source of truth for each encounter may perpetuate a cycle of manual reconciliation, disputes, and delayed settlements.

### BRIEF DESCRIPTION OF DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a block diagram illustrating an example architecture of a healthcare information system, according to some embodiments.

FIG. 2 is a block diagram illustrating a process for generating and/or maintaining a patient graph, according to some embodiments.

FIG. 3 is a block diagram illustrating an architecture for an automated monitoring and action system featuring a sentinel instance, according to some embodiments.

FIG. 4 is a block diagram illustrating an event-driven architecture for automated reporting and compliance monitoring, according to some embodiments.

FIG. 5 is a block diagram illustrating an embodiment of a clinical application for managing collaborative workflows, according to some embodiments.

FIG. 6 is a block diagram illustrating an embodiment of a clinical application that provides a collaborative user interface, according to some embodiments.

FIG. 7 is a diagram illustrating a system for generating a structured note from templates, according to some embodiments.

FIG. 8 is a block diagram illustrating an architecture for natural language processing for incorporating notes, according to some embodiments.

FIG. 9 is a block diagram illustrating an architecture for encounter grouping and claim generation, according to some embodiments.

FIG. 10 is a block diagram illustrating a process for creating a claim for electronic submission, according to some embodiments.

FIG. 11 is a block diagram illustrating an architecture for claim generation, according to some embodiments.

FIG. 12 is a block diagram illustrating an architecture for charge integrity management and modification, according to some embodiments.

FIG. 13 is a block diagram illustrating an architecture for managing the financial lifecycle of a medical claim, according to some embodiments.

FIG. 14A is a flowchart illustrating an exemplary method for processing electronic healthcare transactions, according to some embodiments.

FIG. 14B is a flowchart illustrating an exemplary method for generating one or more claims for electronic submission to a paying party from patient encounters, according to some embodiments.

FIG. 14C is a flowchart illustrating an exemplary method for generating one or more claims from one or more patient encounters, according to some embodiments.

FIG. 15A is a schematic diagram of a neural network model, according to one embodiment;

FIG. 15B is a schematic diagram of a neural network model, according to another embodiment;

FIG. 16 is a schematic block diagram of an example computing system in accordance with an implementation;

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Furthermore, structural and/or other changes may be made without departing from claimed subject matter. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents. Further, it is to be understood that other embodiments may be utilized. Also, embodiments have been provided of claimed subject matter and it is noted that, as such, those illustrative embodiments are inventive and/or unconventional; however, claimed subject matter is not limited to embodiments provided primarily for illustrative purposes. Thus, while advantages have been described in connection with illustrative embodiments, claimed subject matter is inventive and/or unconventional for additional reasons not expressly mentioned in connection with those embodiments. In addition, references throughout this specification to “claimed subject matter” refer to subject matter intended to be covered by one or more claims, and are not necessarily intended to refer to a complete claim set, to a particular combination of claim sets (e.g., method claims, apparatus claims, etc.), or to a particular claim.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the patent application, particular context of description and/or usage provides helpful guidance regarding reasonable inferences to be drawn; however, likewise, “in this context” in general without further qualification refers to the context of the present patent application.

The modern healthcare industry is burdened by significant operational inefficiencies stemming from a fragmented and complex data processing landscape. The lifecycle of a patient encounter, from clinical documentation to financial settlement, may involve numerous disparate systems such as electronic health records (EHRs), billing platforms, payer adjudication systems, and various clearinghouses. Such a lack of integration may create data silos, necessitating redundant manual data entry, which is prone to error and results in inconsistencies between and/or among clinical records and financial claims. Consequently, healthcare providers may face high rates of claim denials, protracted payment cycles, and substantial administrative overhead in managing revenue cycles. This friction may lead to not only revenue leakage and increased operational costs but may also create a frustrating and opaque experience for patients, who often lack clarity regarding their financial responsibilities. The absence of a unified, auditable "single source of truth" that encompasses all patient encounters is a fundamental challenge that prevents real-time, automated, and transparent processing of healthcare transactions.

In an embodiment, medical insurance claims for settlement of services rendered by a service provider may be electronically submitted to a payer or paying party using an electronic claims submission process involving entry of patient and billing information into a practice management system. The entered data may then be converted into a standardized electronic format for secure transmission (e.g., over a communication network) to the payer through a clearinghouse, which helps ensure accuracy and compliance with regulations. Contents of an electronically submitted medical claim may include, for example, patient's date of birth, gender and zip code, National Provider Identifier (NPI) for an attending physician, primary diagnosis code, overall charge for the claim, name of the patient’s insurance company. Additional detail regarding the services rendered may include date of service, procedure code, diagnosis code, National Drug Code (NDC), charge for each service provided or secondary diagnoses or procedures, just to provide a few examples.

In one implementation, a billing system may convert parameters for a claim into a standardized electronic format accepted by insurers. Additionally, an electronically submitted claim may be received at a clearinghouse acting as an intermediary where the claim is scrubbed for errors and routed to one or more appropriate payers. This process creating and settling claims for services rendered to a patient may involve multiple parties, along with human operators that are prone to introducing errors and delays.

Aspects of embodiments described herein are directed to addressing these shortcomings, at least in part, by providing a comprehensive, event-driven healthcare transaction processing platform that unifies clinical and financial workflows through a cohesive, intelligent architecture. In one aspect, particular implementations are directed to processing discrete "event objects" generated to capture events that are relevant to a patient's relationship with a healthcare system and/or facilitate treatment, payment and/or healthcare operations (e.g., including purely administrative events such as IT personnel changing settings, creating accounts, etc.). For example, such an event object may capture creation of a user account for a patient or a service provider, for example. Such an event object may comprise an electronic document stored as signals and/or states in a non-transitory storage medium, and/or as signals encoded in an electronic communication medium. Event objects may express events underlying such event objects as both structured data and unstructured text from clinical notes, signals and/or reports from medical equipment, financial transactions, diagnoses, treatments, just to provide a few examples. Additional events underlying an event object may include user-initiated actions within clinical or other systems, information about laboratory test results, updates to patient's medical history, issuance of medical orders, changes to a patient's care plan, changes in claim processing status, revisions to encounter coding, user credential updates and/or appointment schedule configurations and modifications, just to provide a few additional examples. In an embodiment, event objects may be processed by an advanced language processing engine. In another aspect, deep semantic analysis may be used to identify concepts, classify entities, and evaluate context, thereby transforming contents of event objects into a rich, structured, and interconnected "Patient Graph" that may serve as a dynamic single expression of a patient's attributes relevant to a healthcare system.

According to an embodiment, a patient graph may comprise an electronic document expressing a multi-dimensional, dynamic data model that serves as a central, unified record for a patient within a healthcare information system. A patient graph may be stored and/or expressed in a non-transitory storage medium such as a computer-readable memory device. A patient graph may express not only discrete clinical data points, but may also express intricate relationships between and/or among the discrete clinical points. A patient graph may express attributes such as patient demographics, a longitudinal record of all encounters with various healthcare providers, insurance coverage details, medication lists, known allergies, family medical history, specific diagnoses, treatments administered and/or associated outcomes, test results, observations and/or assessments by a clinician, financial status, insurance coverage, geography, housing status, education status, just to provide a few examples. A particular "graph" structure of a patient graph may allow a computing system to model relationships between and/or among these attributes; for example, a graph structure may enable linking a specific diagnosis to a procedure that was performed to treat the diagnosis, which may in turn be linked to a specific claim that was generated, which may then be linked to a payment event from a paying party. A patient graph may be stateful, meaning it may represent a most current, up-to-date state of a patient's entire clinical and financial journey within a healthcare system.

A patient graph may be configured to be continuously updated in near-real time from a plurality of heterogeneous sources. One source of information may include a stream of event objects generated by computing devices, which can represent actions from clinicians, administrative staff, patients, sentinel instances, or even automated medical equipment. Another information source for updating a patient graph may comprise an output of a transaction processing system used to process event objects (e.g., contextualized data produced by the language processing models and/or contextual analysis models after processing an unstructured clinical note serving as an input for enriching a patient graph).

In particular embodiments, a patient graph may be implemented using a variety of data structures designed to model complex, interconnected information. In one embodiment, a patient graph may be implemented using a native graph database. In a particular implementation, such a patient graph may comprise edges that are weighted to express probabilistic attributes between and/or among nodes connected by the edges. In such a structure, entities such as "Patient," "Encounter," "Diagnosis," and "Claim" may be stored as nodes (or vertices), and the relationships between them are stored as edges. For example, an edge labeled "HAS_DIAGNOSIS" could connect a "Patient" node to a "Diagnosis" node. This structure may facilitate efficient querying of complex relationships, such as finding all treatments from a certain provider. In another embodiment, a patient graph may be implemented using a relational database, where entities are stored in separate tables (e.g., a Patients table, an Encounters table) and relationships may be defined using foreign keys and join tables. In yet another embodiment, a document-oriented NoSQL database could be used, where a patient's entire record, including all encounters, diagnoses, and claims, is stored as a single, complex, nested JSON or BSON document, with relationships being implicit in the document's hierarchical structure. Regardless of the specific implementation, a data structure of a patient graph may be designed to represent a rich, interconnected nature of a patient's relevant healthcare data in a way that is readily machine-readable and queryable by various applications and engines.

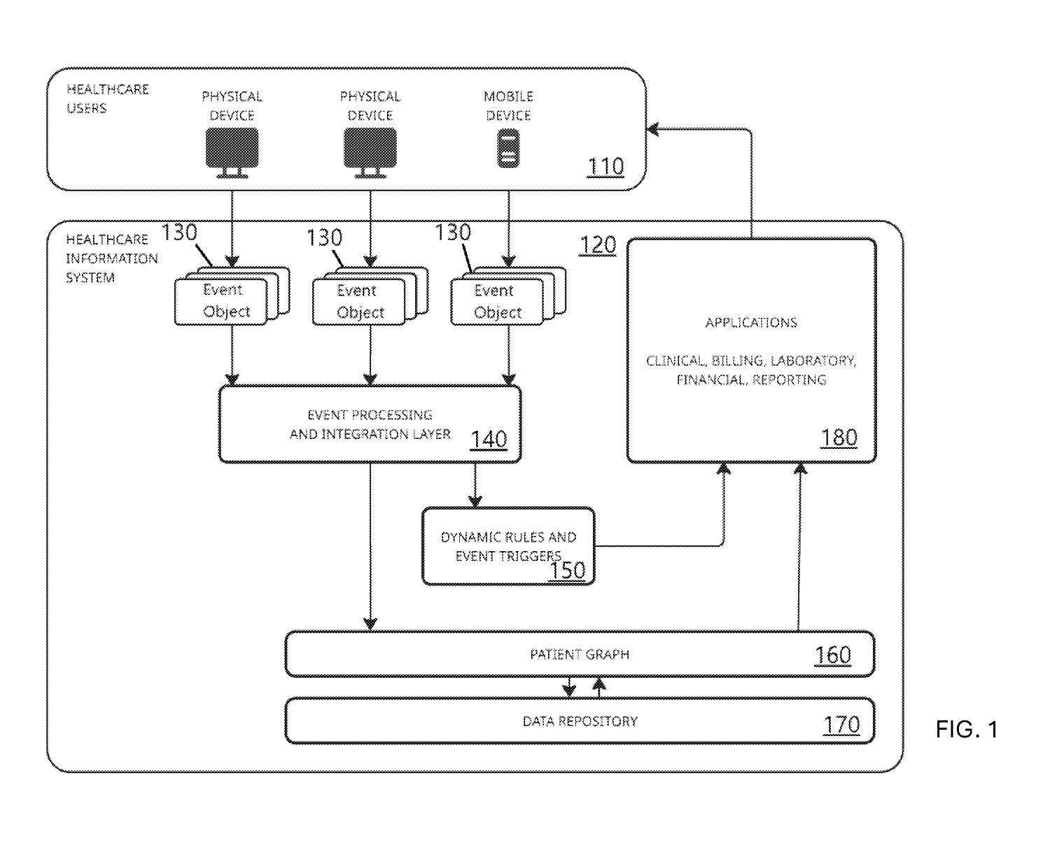

FIG. 1 is a block diagram illustrating an example architecture of a healthcare information system implemented on a server computing device 120, according to some embodiments. One or more client computing devices 110 may be communicatively coupled with server computing device 120. In some embodiments, client computing devices 110 and server computing device 120 may exchange messages over one or more communication networks, such as the Internet, a local area network (LAN), or a wide area network (WAN), according to any suitable network protocol. These protocols may include, but are not limited to, TCP/IP, HTTPS, or other standard or proprietary communication protocols that facilitate secure and reliable message exchange.

Client computing devices 110 may function as any particular device operated by various healthcare users and/or stakeholders to generate one or more event objects 130. For example, a client computing device 110 may comprise a physical device, such as a desktop computer or workstation, operated by a medical service provider to document a clinical encounter. In another example, it may be a mobile device, such as a smartphone or tablet, operated by a patient to provide information. In other embodiments, a client computing device 110 may be operated by a paying party, such as an insurance payer, or a regulatory body. The client computing device 110 may also be integrated with or be part of medical equipment configured to automatically generate event objects 130 based on sensor readings and/or operational status. An event object 130 may be encapsulated in a structured data packet having a payload formatted with a plurality of fields to characterize a particular event. For instance, an event object 130 may include fields for a source identifier indicating its origin, a timestamp, a patient identifier, the relationship to other events, and an event type that categorizes the event, such as 'patient admission,' 'procedure performed,' or 'diagnosis code entered.' Other categories of events, such as the aforementioned user-initiated actions within clinical or other systems, information about laboratory test results, updates to patient's medical history, issuance of medical orders, changes to a patient's care plan, changes in claim processing status, revisions to encounter coding, user credential updates and/or appointment schedule configurations and modifications, may similarly be associated with a descriptive event type to be expressed in an event object 130. In some embodiments, multiple event objects may express attributes of multiple related events, such as in a sequence of events where an initial event triggers and/or spawns a sequence of additional subsequent events. Here, such an event object may include parameters indicating how an underlying event is related to other events (e.g., in a sequence of events) expressed by other event objects.

Upon receipt of an event object 130 from a client computing device 110, server computing device 120 may store the incoming event objects 130 in a memory device, such as a temporary buffer or an ingestion queue, to await further processing. Event processing and integration layer 140 may retrieve one or more stored event objects 130 for processing. Such processing by event processing and integration layer 140 may involve parsing, validating, and transforming parameters parsed from event objects 130 to update a patient graph 160, for example. As pointed out above, patient graph 160 may serve as a structured, relational representation of a patient's attributes and/or state relating to clinical and/or administrative data. Furthermore, event processing and integration layer 140 may initiate processing at one or more applications 180 by making calls to an application programming interface (API) associated with the applications 180.

In some embodiments, dynamic rules and event triggers module 150 may function as an intelligent orchestration engine that automates complex, logic-based workflows within a healthcare information system implemented on server computing device 120. Dynamic rules and event triggers module 150 may dynamically determine rules by, for example, ingesting and synthesizing information from a plurality of sources, which may include internal data such as facility-specific billing guidelines or payer policies, just to provide a few examples. By continuously processing these inputs, dynamic rules and event triggers module 150 may not be reliant on a static rulebook. Rather, dynamic rules and event triggers module 150 may instead generate a set of executable rules that are context-specific and current at the time of processing, thereby adapting to a constantly changing healthcare environment.

Upon the processing of a new event object 130 by the event processing and integration layer 140, dynamic rules and event triggers module 150 may apply the aforementioned dynamically determined rules to initiate downstream actions. Dynamic rules and event triggers module 150 may evaluate parameters parsed from an event object 130 in combination with contextual information retrieved from the patient graph 160 (e.g., from a query to patient graph 160). For example, if an event object 130 containing a new service code is processed, dynamic rules and event triggers module 150 may query patient graph 160 to ascertain a patient's current insurance provider. Dynamic rules and event triggers module 150 may then apply specific rules it has dynamically determined for that particular insurance provider to the new service code. Based, at least in part, on an outcome of this evaluation, dynamic rules and event triggers module 150 may initiate calls to an Application Programming Interface (API) for the execution of one or more applications 180. For instance, if the rules indicate that pre-authorization is required for a procedure under the patient's current plan, dynamic rules and event triggers module 150 may initiate a compliance-checking application; otherwise, dynamic rules and event triggers module 150 may directly initiate a billing application. This process may enable automation of complex, context-dependent decision-making that would otherwise require manual intervention.

According to an embodiment, server computing device 120 may be configured to be dynamic and context-aware. In some embodiments, the execution of a specific application 180 via the API may be based on parameters maintained in patient graph 160. For example, after processing a new event object 130 to update patient graph 160, event processing and integration layer 140 may query the patient graph 160 to check a patient's current insurance status or encounter history. Based on the retrieved parameters, the system may then trigger a specific billing workflow or a clinical alert within the applications 180, ensuring that subsequent actions are contextually appropriate. Applications 180, in turn, can interact with dynamic rules and event triggers 150 to orchestrate complex workflows, with significant actions being recorded in an audit trail 190 (e.g., that is also stored in the data repository 170 and linked to the patient graph 160).

Implementation of event processing and integration layer 140, dynamic rules and event triggers 150 and patient graph 160, working collectively to process event objects 130, may provide a specific improvement in computing technology by transforming the computer from a collection of passive, siloed data repositories into an active, context-aware transaction processing engine. This may address the technical problem of operational inefficiencies caused by fragmented data systems that require a human user to act as the integration point between different software applications. Specifically, event processing and integration layer 140 may retrieve and normalize disparate and asynchronous event objects 130 into a unified data stream that updates patient graph 160. This may create a specific, structured, and interconnected data model that represents the real-time state of a patient across all encounters, a task the computer could not previously perform. Dynamic rules and event triggers module 150 may then programmatically and autonomously evaluate patient graph 160, initiating subsequent workflows in applications 180. By creating this cohesive, closed-loop architecture, the system may programmatically link a received clinical event to a specific, rule-based administrative action (e.g., alerts and tagging patients) without human intervention, thereby functioning as a more efficient and reliable computing tool specifically adapted for the complex, event-driven nature of healthcare transactions.

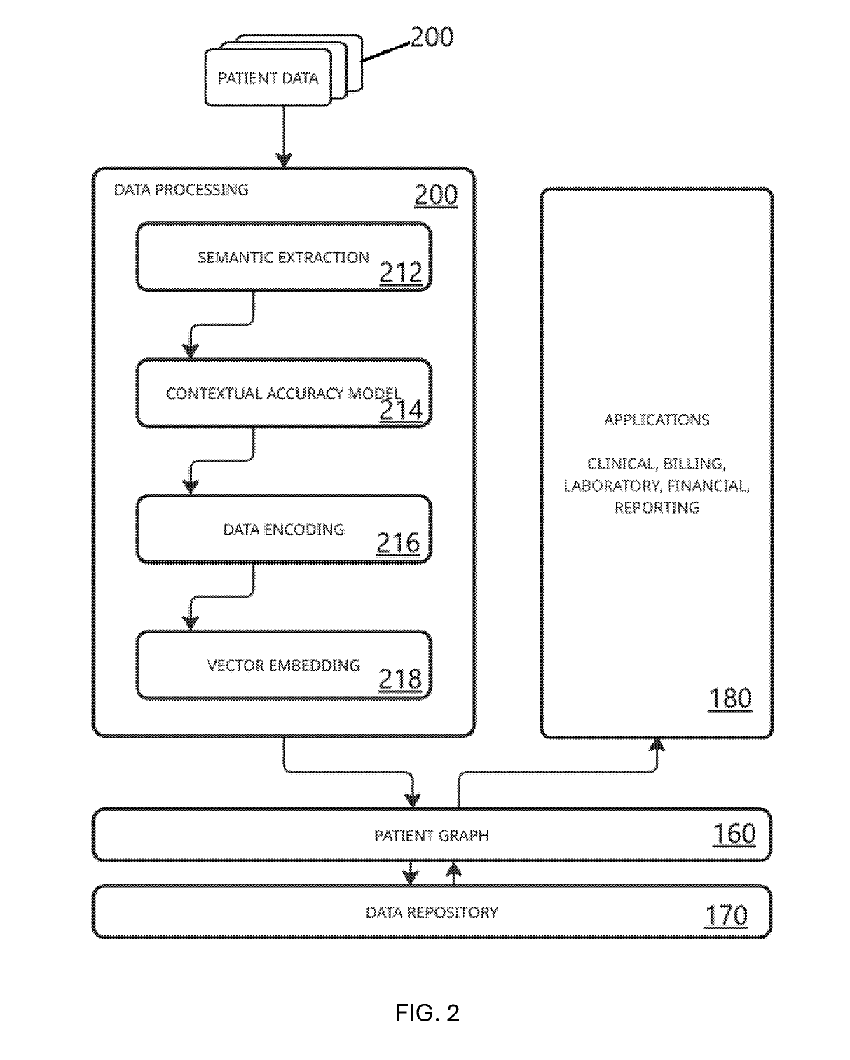

FIG. 2 is a block diagram that illustrates a process for generating and maintaining a patient graph 160, according to some embodiments. Patient graph 160 may comprise a central component of a healthcare information system, providing a dynamic and comprehensive data structure that expresses attributes and/or a state of a patient. In some embodiments, patient graph 160 may maintain a unified, structured record characterizing a patient within the healthcare system. Patient graph 160 may also aggregate and normalize patient data from a plurality of data sources, including external healthcare systems and multiple healthcare institutions operating within the ecosystem of a healthcare information system. Patient graph 160 may include, but is not limited to, a comprehensive history of the patient's encounters with healthcare providers, details of insurance coverage, current and past medications, family medical history, diagnoses, treatments, and associated outcomes, just to provide a few examples. By structuring attributes and/or data in a relational or graph format in patient graph 160, a healthcare system may efficiently traverse and query complex relationships within a patient's attributes.

A patient graph 160 may be generated and updated based, at least in part, on patient data 210, which may be constituted from a plurality of different sources. These sources may include, for example, processed event objects 130 (as described with reference to FIG. 1), which may represent and/or express parameters representative of discrete clinical and/or administrative actions relating to a patient in a healthcare system. For example, an event object 130 may originate from a clinician's interaction with an EHR, a lab result from a diagnostic machine, or a billing update, just to provide a few examples. Additionally, patient data 210 may include parameters and information furnished directly by a patient, for instance, through a patient portal or a mobile application, or from third-party data streams. Other sources of patient data 210 may include data imported from multiple external health systems, records from pharmacy systems, and data from health information exchanges.

As shown in FIG. 2, a data processing block 200 may process incoming patient data 210 through a processing pipeline before patient data 210 affects a state of patient graph 160. Such a pipeline may include a semantic extraction module 212, which may be configured to identify key clinical and financial concepts and entities from structured or unstructured data. A contextual accuracy model 214 may then be applied to validate and interpret the parameters extracted from event objects' 130 information within its proper clinical context, for example, by distinguishing between and/or among a diagnosis, treatment and/or a family history item. Subsequent processing by a data encoding module 216 and a vector embedding module 218 may transform contextualized information into a structured, machine-readable format, such as numerical vectors suitable for machine learning and relational analysis, just to provide a few examples. An output of such a processing pipeline may provide a set of derived parameters that are used to affect or update a state of the patient graph 160.

A state of patient graph 160 may also be affected by, or defined in conjunction with, items stored in a data repository 170. Data repository 170 may store the underlying raw data, historical states of patient graph 160, or large-scale data sets, while patient graph 160 may represent an active, relational model of that data. Data repository 170 and patient graph 160 may be communicatively coupled, allowing for data to be archived, retrieved, and used to reconstruct and/or enrich patient graph 160 as desired.

In an embodiment, patient graph 160 may be tightly coupled with both upstream processing of event objects 130 and downstream execution of applications 180. Processing of event objects 130 may directly drive updates to patient graph 160, creating a real-time link between events (e.g., clinical treatment, diagnosis, financial event, etc.) and a state of a patient's patient graph 160. Concurrently, applications 180, which may include clinical, billing, and reporting functionalities, may be configured to query patient graph 160, often via an application programming interface (API), to retrieve parameters enabling proper execution. Such a tight coupling may enable applications 180 to operate on the most current, contextually accurate, and semantically rich representation of a patient's state as maintained in patient graph 160.

A tight integration and coupling of patient graph 160 with a real-time processing of event objects 130 for the execution of clinical and administrative applications in applications 180 may provide a specific improvement in computing technology by addressing the operational inefficiencies and/or latencies caused by fragmented data systems. In some systems, clinical and administrative data reside in separate, siloed databases. A computer may act as a passive repository for this disconnected information, requiring a human user to manually query multiple systems and perform the cognitive task of correlating disparate data points to gain a complete picture of a patient's encounter before taking an action. In contrast, the described architecture may configure a computing device to function as an active data synthesizer. As event objects 130 are processed, patient graph 160 is not merely appended with new parameters; its relational structure may be dynamically updated in real-time. This may transform patient graph 160 into a comprehensive, stateful, and machine-readable model of the patient's entire encounter lifecycle. Consequently, if a clinical or administrative application is executed, it may query this single, unified patient graph 160 to immediately retrieve a complete, context-aware dataset without needing to perform complex, cross-system joins and/or employ a human user to bridge informational gaps. By creating this integrated patient graph 160 that is programmatically updated and queried by applications, an electronic healthcare system may function as a more efficient, reliable and low-latency computing tool, one specifically adapted to overcome the data fragmentation inherent in complex healthcare environments. Additionally, patient graph 160 embodied as signals and/or states stored in a non-transitory memory may be physically transformed as an article to adapt in real-time responsive to events that affect patient’s status, which enhances the usefulness of a computing system in processing transactions involving the patient with reduced latency and fewer errors.

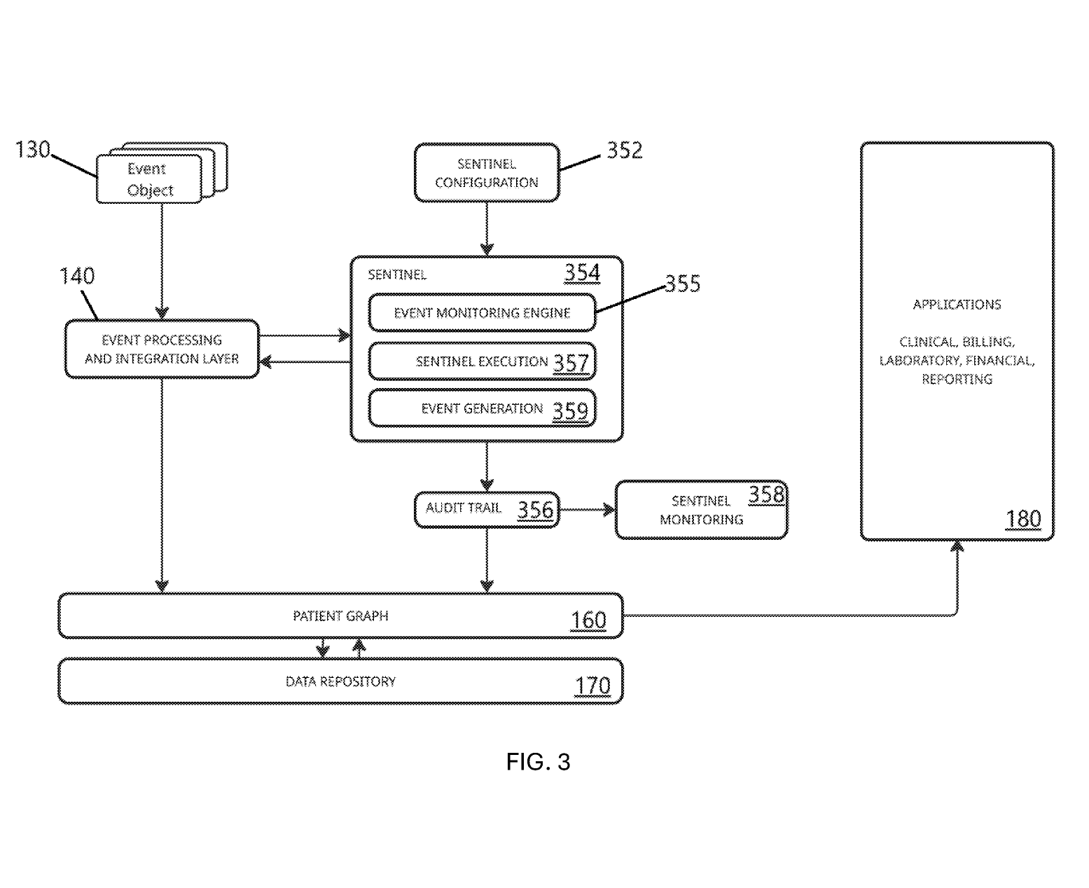

FIG. 3 is a block diagram illustrating an architecture of an automated monitoring and action system featuring a sentinel instance 354, according to some embodiments. Server computing device 120 may be configured to host one or more sentinel instances 354. Behavior, trigger conditions and/or actions of sentinel instance 354 may be governed by a corresponding sentinel configuration 352. Sentinel configuration 352 may be defined by a set of pre-defined rules, user-configurable parameters, executable computer code, or a combination thereof, which may dictate how sentinel instance 354 is to respond to specific events and/or states within a healthcare system.

In some embodiments, sentinel instance 354 may operate as an autonomous agent configured to perform a wide range of automated actions. Such automated actions may be performed responsive to processing of event objects 130 by the event processing and integration layer 140. For example, upon detection of an event object 130 indicating a new critical diagnosis for a patient, sentinel instance 354 may autonomously initiate a set of diagnostic or treatment procedures, such as automatically ordering requisite lab tests. In another example, sentinel instance 354 may automatically alert a specific service provider or care team member in response to an event object 130 containing an out-of-range lab value. In yet another embodiment, sentinel instance 354 may initiate a financial transaction, such as a processing of a co-payment, upon detection of an event object 130 signifying the completion of a patient check-in process.

To ensure transparency and traceability, an audit trail 356 may maintain a persistent and immutable record of actions initiated by sentinel instance 354. Audit trail 356 may log details such as the triggering event, the action taken, the timestamp of the action, and the outcome, for example. A sentinel monitoring module 358 may provide an interface within the applications 180 for users to review the activities recorded in the audit trail 356.

Functionality of sentinel instance 354 may be carried out by one or more internal components. Event monitoring engine 355 may be configured to process signals from the event processing and integration layer 140. The event monitoring engine 355 may, for example, continuously compare parameters of incoming processed event objects 130 against trigger conditions defined in sentinel configuration 352. If a particular condition is met, a sentinel execution module 357 may be invoked, which may in turn direct an event generation module 359 to initiate a configured action. Such action may involve creating a new event object, invoking a function in the applications 180 and/or making an API call to an external system, for example.

Furthermore, a state of patient graph 160 may be updated to reflect actions initiated by sentinel instance 354. Information recorded in audit trail 356 may be integrated into patient graph 160, ensuring that the patient's unified record is current and accurately represents system-automated activities. For example, if sentinel instance 354 orders a lab test, the patient graph 160 may be updated to include this new order, linking the new order to the patient and triggering a clinical event. This may maintain the patient graph 160 as a comprehensive single source of truth for all manual and automated aspects of a patient's care journey.

In some embodiments, sentinel instance 354 may be implemented as a hybrid system that combines a deterministic expert system with an adaptive, trained computing model. Such a mixture may allow sentinel instance 354 to handle both explicit, hard-coded logic and complex, nuanced patterns learned from data, providing a more powerful and flexible automation capability.

An expert system component of sentinel instance 354 may comprise a knowledge base and an inference engine. Such a knowledge base may store a set of explicit, human-defined rules, often in an "IF-THEN" format. These rules may encode well-defined business logic, clinical protocols, or regulatory requirements, such as payer policies and/or facility guidelines. For example, a rule in such an expert system might state: "IF an event object indicates a specific surgical procedure was performed, THEN automatically add a corresponding charge for surgical supplies to the claim." This part of sentinel instance 354 may provide transparent, auditable, and reliable execution of known, deterministic tasks.

A trained computing model component of sentinel instance 354 may comprise a neural network, a decision tree and/or a large language model to provide adaptive intelligence. Such a model may be pre-trained on a vast corpus of text and fine-tuned on medical and clinical literature (e.g., using a self-supervised learning), followed by training on more focused training sets (e.g., using supervised learning), for example, to identify complex patterns, correlations, or anomalies that are not easily captured by simple "IF-THEN" rules. For example, such a trained model may analyze a combination of a patient's vitals, lab results, and demographic data to predict a likelihood of an impending adverse event.

In an embodiment, expert system and trained computing model components of sentinel instance 354 may work in concert. For example, a trained computing model may analyze incoming parameters parsed from an event object 130 and generate a probabilistic output, such as a risk score or a classification. This output may then serve as a dynamic input for an expert system. For instance, a trained model component may predict a "high probability of claim denial" based on a complex pattern of diagnosis and treatment codes. An expert system component may then act on this prediction with a deterministic rule: "IF 'Claim Denial Probability' > 90%, THEN flag the claim for manual review AND notify the billing department." In this hybrid implementation, the sentinel instance 354 may not be limited to reacting to simple events and may intelligently respond to subtle, complex patterns, making it a far more effective and efficient tool for automating healthcare workflows. In one particular implementation, event monitoring engine 355 may track events expressed in multiple event objects 130 to assess a situation and initiate an appropriate response. For example, event monitoring engine 355 may associate events in multiple different event objects 130 according to time, space, location and/or other attributes to be in a group of events for which a particular response may be appropriate. Such a response may then be duly executed by sentinel execution 357 and event generation 359, for example.

Implementation of the sentinel instance 354 may provide a specific improvement in computing technology by transforming the healthcare information system from a passive data repository into a proactive, autonomous workflow engine. This may address the technical problem of operational inefficiencies caused by fragmented systems that require human users to manually monitor for critical events and then initiate appropriate actions. For example, sentinel instance 354 may be configured as an autonomous agent with an event monitoring engine 355 that continuously and programmatically evaluates event data as it is processed, without waiting for a direct user query. Upon detecting a trigger condition defined in its sentinel configuration 352-such as a critical lab value or a specific new diagnosis-the sentinel instance 354 is configured to autonomously execute a pre-defined action, such as generating an alert to a provider and/or initiating a follow-up workflow. By creating this tightly integrated, automated "sense-and-respond" mechanism directly within the computing architecture, an electronic healthcare system may perform complex, rule-based monitoring and action initiation tasks that would otherwise rely on the non-scalable and error-prone process of human intervention, thereby functioning as a more efficient and reliable computing tool for managing real-time healthcare transactions.

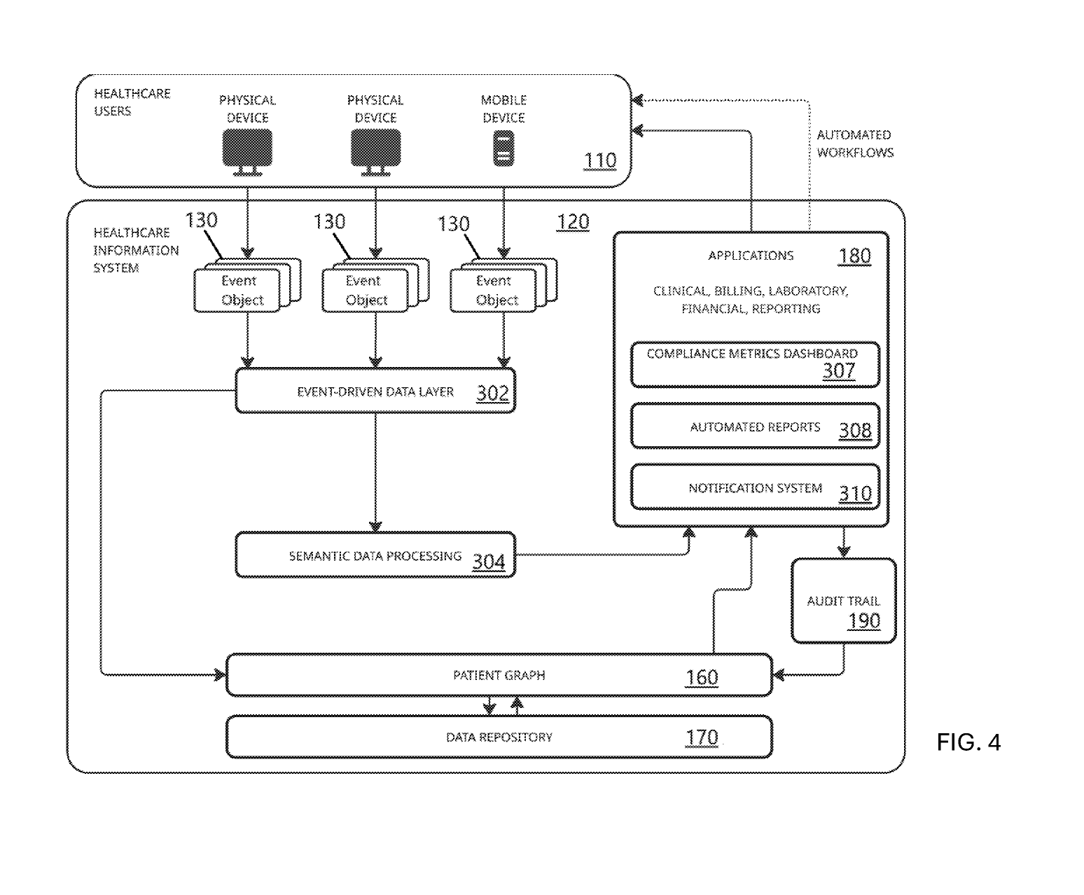

FIG. 4 is a block diagram illustrating an event-driven architecture for automated reporting and compliance monitoring within a healthcare information system implemented by server computing device 120, according to some embodiments. For example, event objects 130 generated by healthcare users may be processed to provide real-time insights and automated outputs to support compliance and related reporting. As shown, event objects 130 originating from client computing devices 110 may be received by an event-driven data layer 302. In some embodiments, event-driven data layer 302 may comprise a component and/or functional aspect of the event processing and integration layer 140 (FIG. 1) and may be configured to manage the ingestion and queuing of incoming event objects 130 for further processing. Event-driven data layer 302 may handle event objects 130 in a structured and timely manner, preparing event objects 130 for semantic analysis, for example.

Parameters parsed from event objects 130 may be subsequently processed by a semantic data processing module 304. Semantic data processing module 304 may perform deep semantic analysis on parameters parsed from event objects 130, extracting and structuring key parameters related to clinical activities, billing information, or other relevant parameters. In some embodiments, the semantic data processing 304 may process event objects 130 to provide structured, validated inputs to an Application Programming Interface (API). This API may then initiate calls to applications 180 to facilitate automated compliance reporting.

An output of the semantic data processing module 304 may also drive a notification system 310. Notification system 310 may generate alerts, notifications and/or signals based, at least in part, on the occurrence of predefined events or the identification of specific data patterns, for example. In some embodiments, notification system 310 may facilitate generation of automated reports 308 by triggering a report generation process in response to a specific alert. For instance, a notification indicating a potential compliance breach may trigger an automated report 308 detailing the incident. Additionally, notification system 310 may facilitate real-time update of a compliance metrics dashboard, which may be part of the applications 180, by pushing new data points or metrics as they are identified through semantic data processing 304. Aspects of this process may be recorded in an audit trail 190, and used to update patient graph 160.

Automated reports 308 and compliance metrics dashboard 307 generated by applications 180 may facilitate compliance reporting capabilities. In various embodiments, such compliance reporting may be directed to a number of different entities. For example, reports may be formatted for submission to government agencies to demonstrate adherence to regulatory requirements and quality measures. In other cases, reports may be generated for paying parties and/or insurance companies to provide evidence of contractual compliance, utilization data and/or adherence to specific care protocols.

The combination of event-driven layer 302, semantic data processing 304, automated reports 308 and notification system 310, as shown in FIG. 4, may provide a specific improvement in computing technology by creating a real-time, automated compliance monitoring and reporting system that overcomes the operational inefficiencies of manual, retrospective data analysis inherent in fragmented healthcare systems. For example, event-driven layer 302 may retrieve event objects 130 in near-real time to be processed by the semantic data processing 304. Semantic data processing module 304 may perform contextual analysis to understand the compliance implications of an event, rather than merely storing associated data. This may transform a computing device from a passive data repository, which would require a human analyst to manually query and correlate data for compliance checks, into a proactive monitoring engine. Based, at least in part, on an output of semantic data processing 304, notification system 310 may be automatically triggered to generate an alert, initiate creation of an automated report 308 and/or update a compliance metrics dashboard 307 in real time. By creating this tightly integrated, closed-loop architecture that programmatically links semantic data understanding with automated output generation, the system functions as a more efficient and reliable computing tool specifically adapted for the complex task of continuous compliance monitoring.

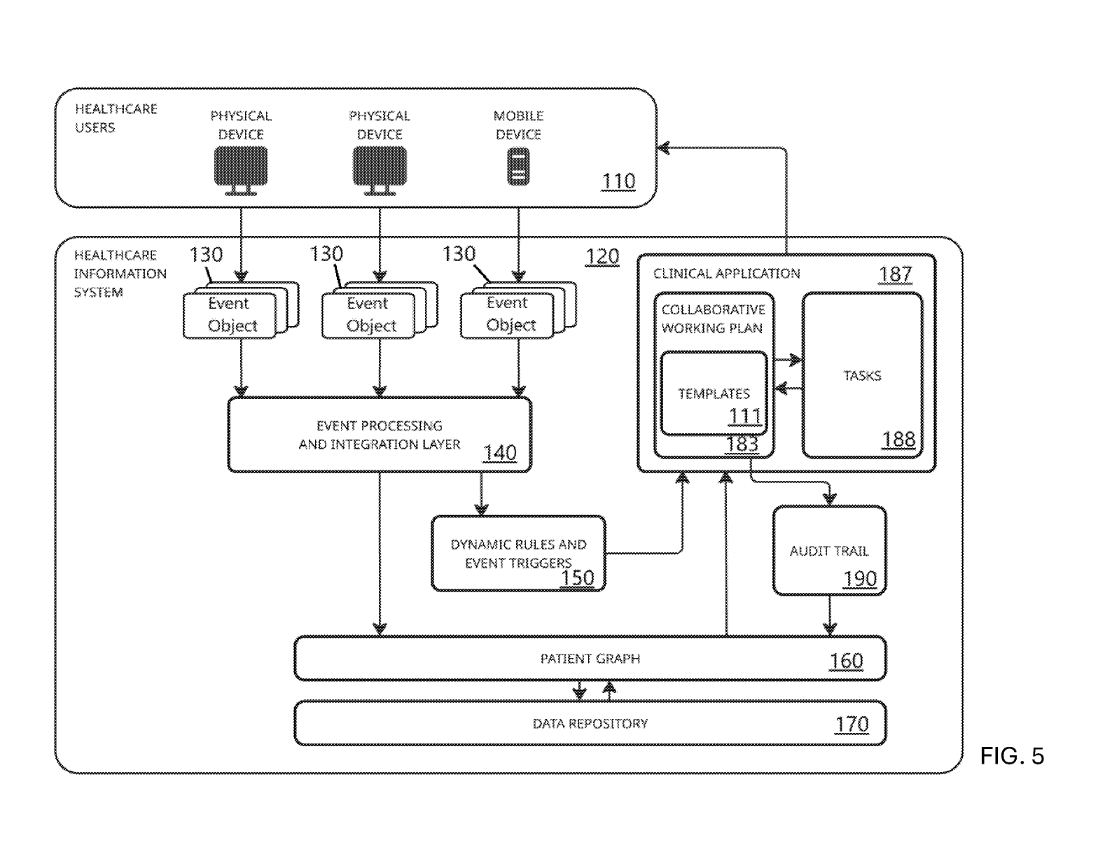

FIG. 5 is a block diagram illustrating an embodiment of a clinical application 187 hosted on the server computing device 120. Clinical application 187 may serve as a primary interface for healthcare users and may leverage parameters maintained in patient graph 160 to orchestrate complex clinical and/or administrative workflows. In some embodiments, the clinical application 187 may facilitate collaboration between and/or among a plurality of parties involved in a patient's care journey. These parties may include, but are not limited to, patient's various medical service providers, and paying parties. These parties may include, but are not limited to, patients, patients' guardians, patients' guarantors, various medical service providers, healthcare administrative staff, and paying parties. For instance, in one example, such collaboration may be coordinated between a primary care physician and one or more specialists. By providing all authorized parties with access to a shared and consistent set of data and workflows derived from the patient graph 160, the clinical application 187 may ensure that stakeholders are operating from the same unified information, thereby reducing miscommunication and streamlining care coordination.

Aspects of this collaborative functionality may be enabled by a collaborative working plan 183. Collaborative working plan 183 may define one or more templates 111 to coordinate activities and responsibilities between and/or among different parties. Such a template may pre-define a sequence of actions, required documentation and/or responsible parties for a specific clinical scenario, such as a post-operative recovery plan or a chronic disease management protocol, for example. Upon initiation of a collaborative working plan 183, a selected template may automatically generate and assign a plurality of tasks 188 to appropriate parties. For example, a selected template may assign a task to a patient to report daily symptoms via their mobile device, a task to the primary physician to review those symptoms and/or a task to the specialist to conduct a follow-up consultation after a set period, just to provide a few examples.

In one example, initiation of a collaborative working plan 183 may be triggered automatically by dynamic rules and event triggers module 150 in response to a new event object 130. In another example, collaborative working plan 183 may be initiated manually by a user through the clinical application 187. Interactions with the collaborative working plan 183 and the completion of tasks 188 are recorded as new events, which may subsequently be used to update patient graph 160 and may be logged in audit trail 190, ensuring a complete and auditable history of a coordinated care process.

According to an embodiment, implementation of collaborative working plan 183 in combination with the generation of tasks 188 may provide a specific improvement in computing technology by creating an automated, multi-party workflow orchestration engine. This may address operational inefficiencies in healthcare, which stem from fragmented systems that require manual, out-of-band communication (such as phone calls or emails) to coordinate care between and/or among different providers or departments. Specifically, collaborative working plan 183 may function as a machine-executable template that defines a sequence of actions for a given clinical scenario. Upon initiation, collaboration working plan 183 may be instantiated to generate and/or distribute a plurality of discrete, trackable tasks 188 to appropriate parties, such as a primary physician and a specialist. This may transform a computing device from a passive repository for storing static care plan documents into an active workflow coordinator that manages a state and assignment of each action in the process. By creating a single, system-managed process that programmatically coordinates the actions of multiple, distinct parties, this architecture represents a more efficient and reliable computerized process for executing complex healthcare transactions.

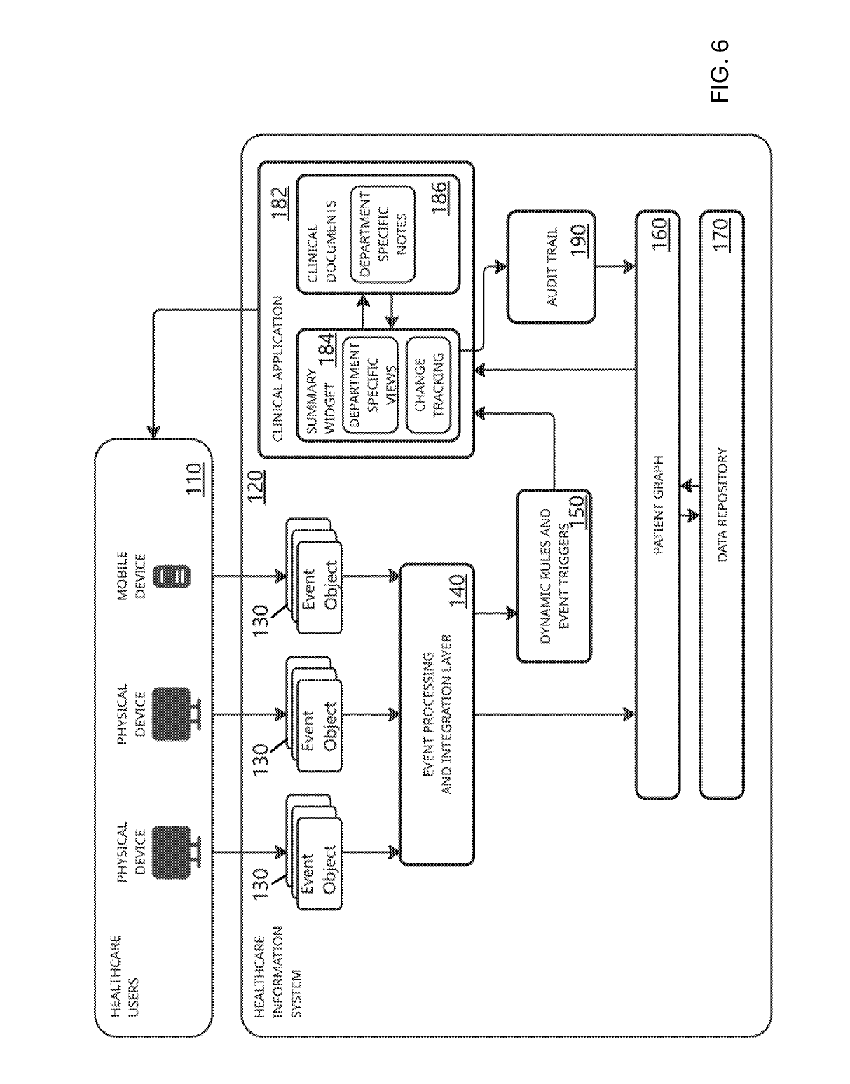

FIG. 6 is a block diagram illustrating an embodiment of a clinical application 182 that provides a collaborative user interface, according to some embodiments. The clinical application 182 may serve as a primary interface for various healthcare users to interact with data managed by a healthcare information system implemented on a server computing device 120. In some embodiments, clinical application 182 may be configured to facilitate collaboration between and/or among a plurality of different parties, such as patients, various medical service providers, healthcare administrators and/or paying parties, just to provide a few examples. This collaboration may be enabled through the configurable nature of the clinical application 182 (e.g., accessible via a displayed dashboard), as well as through a summary widget 184, which may function as a dynamic and customizable summary. Summary widget 184 (e.g., within a displayed dashboard, not shown) may be configured to express department-specific and/or party-specific summaries of a patient's data, which may be retrieved from the patient graph 160, for example. In a particular example, a cardiologist interacting with the summary widget 184 may be presented with a summary that prioritizes cardiac-related events, medications, and lab results, while a billing specialist may be presented with a summary that emphasizes billable procedures and encounter dates. Such a role-based filtering may ensure that each party is presented with the most relevant information, reducing cognitive load while maintaining a shared context derived from the same underlying patient graph 160. Furthermore, the summary widget 184 may be configured to provide change tracking functionality, for instance, by highlighting data elements that have been added or modified since the user's last review, with such changes being logged in audit trail 190. According to an embodiment, summary widget 184 may be included as part of a patient chart (e.g., displayable on a tablet screen) which is customizable in some respects. For example, such a patient chart may be customizable to provide views for different parties such as physician, nurse or specialist (e.g., cardiologist).

To provide a comprehensive and context-rich overview, the summary widget 184 may be further configured to integrate information from a plurality of clinical documents 186 (which may be expressed in electronic documents). Clinical documents 186 may comprise, for example, a collection of notes, reports, and other documents generated by different departments and/or providers. Summary widget 184 can be configured to automatically identify, retrieve, and display relevant department-specific notes within its user interface. For example, the summary widget 184, while displaying a view for a consulting specialist, may be configured to automatically pull in and display the most recent progress notes from the referring primary care physician and the latest diagnostic reports from a radiology department. Such an integration may provide a consolidated summary of a patient's status without requiring the user to manually locate and open numerous individual documents, thereby improving efficiency and supporting more informed clinical decision-making.

Implementation of summary widget 184 in combination with an integration of clinical documents 186 may provide a specific improvement in computing technology by overcoming the operational inefficiencies caused by fragmented data systems that force users to manually locate and synthesize information from numerous disparate sources. Specifically, the summary widget 184 may automatically query, retrieve, and intelligently integrate relevant data from a plurality of clinical documents 186-such as department-specific notes-and present a single, consolidated view tailored to the user's specific role or department. This may transform a computing device from a passive file-serving system, which requires a human user to perform the cognitive task of finding and correlating information, into an active data synthesis engine that programmatically performs this aggregation. By performing the data aggregation and contextual filtering tasks automatically, the system reduces user cognitive load and the time required to gain a comprehensive understanding of a patient's status, thereby functioning as a more efficient and effective information processing tool specifically adapted for the complex healthcare environment.

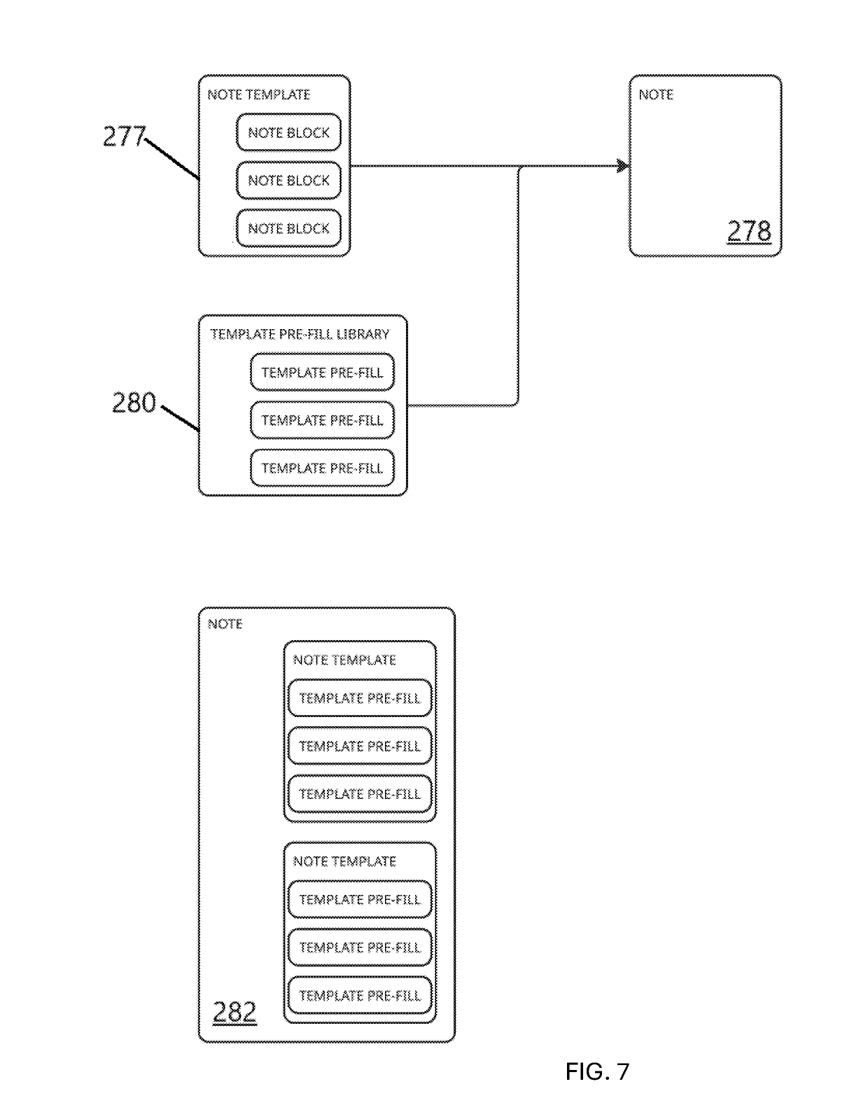

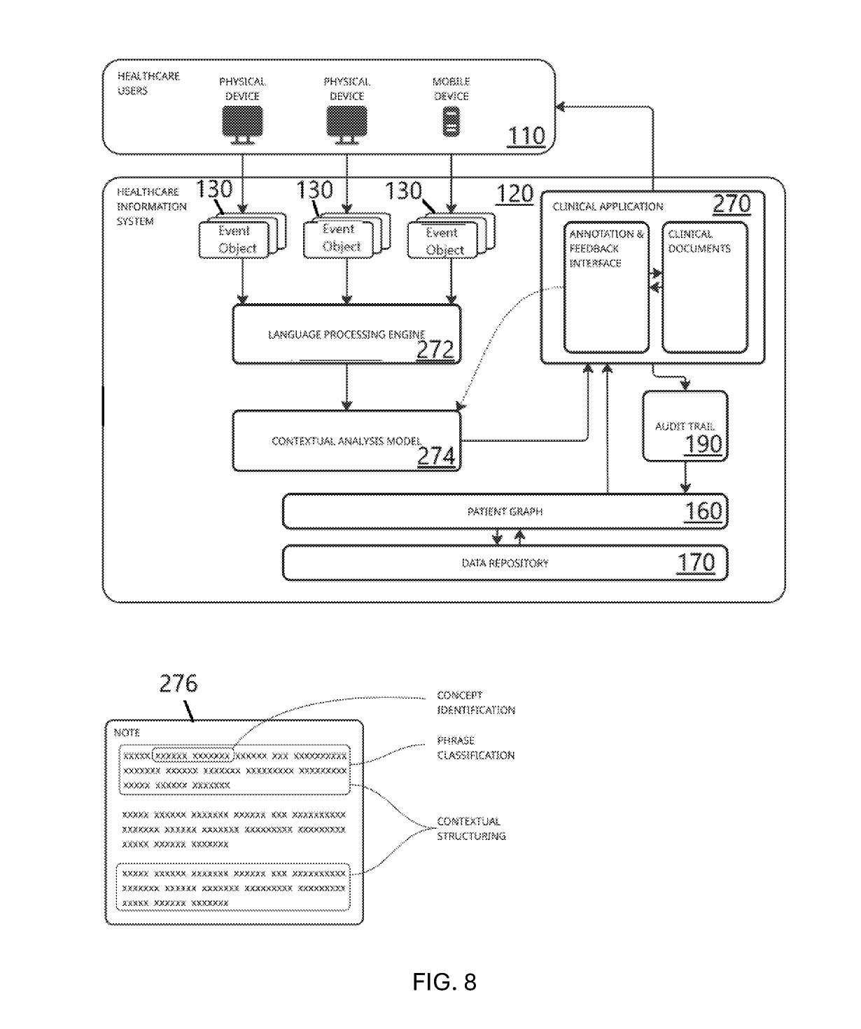

FIG. 7 is a block diagram illustrating prefill fields for a clinical note, and FIG. 8 is a block diagram illustrating an architecture for natural language processing of clinical notes, according to some embodiments. A template of pre-fill fields shown in FIG. 7 may enable configuration of options for clinician-specific pre-filled fields to enhance note-taking efficiency. A significant challenge in healthcare data processing is that raw information received in event objects 130, particularly from clinical documentation, is often unstructured, ambiguous, and/or lacking in context. Additionally, a clinical note may mention a symptom without specifying whether the symptom is of a current complaint, part of a patient's family history, or a resolved issue.

To make sense of clinical documentation that is unstructured, ambiguous and/or lacking in context, event processing and integration layer 140 may implement a language processing engine 272 and a contextual analysis model 274, as shown in FIG. 8. The language processing engine 272 may parse items and text from unstructured event objects 130. Such parsed items and text may then be processed by contextual analysis model 274, which may apply natural language understanding techniques to provide context and/or fidelity to parameters parsed from event objects 130. This process may refine raw data into structured parameters that can be passed through an Application Programming Interface (API) to be further processed by a particular clinical application 270 and/or used to update patient graph 160, for example.

In some embodiments, processing performed by the contextual analysis model 274 may be affected, at least in part, by inputs received from an annotation and feedback interface, which may be implemented within the clinical application 270. This may create a human-in-the-loop feedback system where a user can correct or enhance the model's understanding. The annotation and feedback interface may be configured to receive inputs via various means, including a graphical user interface (GUI) on a physical device, a microphone for voice commands via a voice user interface (not shown), or another suitable input device.

The annotation and feedback interface may receive inputs for providing greater context and/or fidelity to the system using notes, such as note 276. For example, a user may receive an unstructured event object containing the dictated phrase, "The patient's mother had breast cancer." The language processing engine 272 may initially identify "breast cancer" as a clinical concept. Using the annotation and feedback interface and note 276, a user may then provide greater fidelity by performing a concept identification on "breast cancer," applying a phrase classification of "Family Medical History," and further defining the contextual structuring as "Maternal." This structured feedback provides high-fidelity data to the patient graph 160 and may also be used to train and/or refine the contextual analysis model 274, improving its accuracy for processing future, similar events.

As shown in FIG. 7, to facilitate the efficient creation of structured inputs, a note 278 or 282 may be formed according to predefined templates. A note template 277, which may itself be composed of smaller note blocks, can be obtained from a template pre-fill library 280. In an embodiment, pre-fill library 280 can automatically populate fields in note template 277 with known data from the patient graph 160, reducing manual entry and ensuring consistency while still allowing for user annotation and feedback to add further structure and fidelity.

In some embodiments, the contextual analysis model 274 may be implemented using a language model, such as a transformer-based neural network model that has been pre-trained on a vast corpus of text and fine-tuned on medical and clinical literature. The inherent capabilities of a language model in understanding grammar, semantics, and context make it particularly well-suited for the tasks performed by this model. In such an implementation, when the language processing engine 272 parses unstructured text from an event object 130, the parsed text may be provided as a prompt to the language model.

In this context, a language model may then perform several functions. First, such a language model may execute advanced semantic extraction by identifying clinical entities within the text (e.g., medications, symptoms, diagnoses, procedures). Second, such a language model may analyze surrounding words and sentence structure to determine a context of those clinical entities. For instance, given the phrase "patient denies chest pain, but mother had hypertension," the language model can differentiate that "chest pain" is a negated, current symptom for the patient, while "hypertension" is an affirmed condition related to family history.

Furthermore, such a language model-based contextual analysis model 274 may be integrated with the annotation and feedback interface. If an initial analysis of the language model is presented to a user for verification, any corrections or annotations made by the user can be collected as high-quality training data. This data can then be used to periodically fine-tune the language model, adapting its performance to the specific terminology and documentation patterns of a particular clinical specialty or institution. This may create a self-improving system where the contextual analysis model 274 becomes progressively more accurate and efficient at converting unstructured clinical narratives into the structured, high-fidelity data to update patient graph 160. In a particular example, use by a specific clinician may incrementally improve the accuracy of contextual analysis model 274 for every subsequent user. Additionally, with usage of contextual analysis model 274 overtime by specific clinicians, contextual analysis model 274 may improve over time particularly for subsequent uses by the specific clinicians.

A combination of the annotation and feedback interface of the clinical application 270, the unstructured note 276, pre-filled fields from pre-fill library 280 and the note template 277 may provide a specific improvement in computing technology by creating a human-in-the-loop data refinement system. This may address the technical problem of operational inefficiencies arising from the complex and often ambiguous nature of unstructured clinical data, which some computing systems cannot reliably process for automated workflows. Specifically, if ambiguous parameters within an event object 130 are encountered, the annotation and feedback interface may enable improved context and fidelity. Instead of relying on unstructured user corrections, the annotation and feedback interface may utilize predefined fields of note 276-such as concept identification, phrase classification, and contextual structuring. This may transform human feedback into structured, high-fidelity data that is immediately machine-readable and actionable by the system's contextual analysis model 274. The use of note template 277 pre-filled fields from pre-fill library 280 may further enhance this process by pre-populating known data, streamlining a user's annotation task. By integrating this structured feedback loop directly into the data processing workflow, the system may function as a more effective computing tool, one that can dynamically and reliably convert ambiguous, unstructured data into the precise, context-aware parameters for automated transaction processing.

In this context, a patient "encounter," as referred to herein, is any interaction between a patient and a healthcare service provider where medical services, treatment, diagnosis and/or advice are delivered and documented. This can include in-person visits, telehealth appointments, tests performed, or any other form of healthcare interaction, just to provide a few examples. In an embodiment, a patient's encounters with a medical service provider may be settled for payment by submission of a separate claim for each encounter. For example, for each of the patient's encounters with a medical service provider, a single claim may be prepared for electronic submission to a paying party.

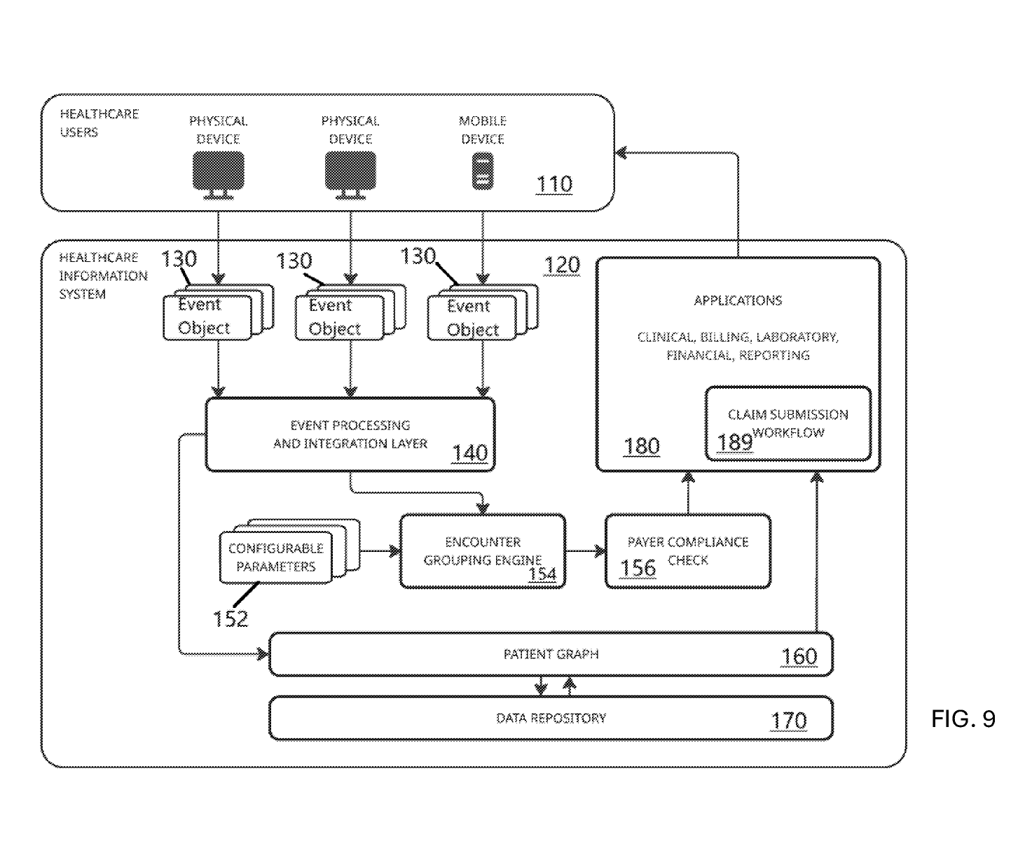

In an embodiment, for any particular medical patient, server computer device 120 may maintain a record of encounters of the particular medical patient with medical service providers as signals and/or states in a memory device formatted in electronic documents as "encounter objects." For example, each such encounter object may comprise parameters to express an encounter type (e.g., type of medical service as ED or outpatient, treatments received), patient, service provider, reason, status, date and/or time of service, just to provide a few examples. According to an embodiment as shown in FIG. 9, parameters of multiple encounters of a patient with one or more medical service providers may be combined and/or bundled for settlement using claim submissions that need not correspond one-to-one with the multiple encounters.

FIG. 9 is a block diagram illustrating an architecture for patient encounter grouping and claim generation, according to some embodiments. In one implementation, encounter grouping engine 154 may facilitate a workflow in which multiple patient encounters may be processed to generate one or more claims for electronic submission to a paying party. In one embodiment, encounter objects embodied as signals and/or states stored in a non-transitory memory may transformed to a more useful expression of an encounter group, which may facilitate formation of electronic medical claims submitted with greater accuracy, greater transparency and reduced latency. In a particular implementation, encounter object may be maintained as electronic documents stored as part of configuration parameters 152 or on a separate memory device (not shown).

Encounter objects to express attributes of a patient encounter may be formed by one or more methods such as, for example, receipt of encounter objects from an external source and/or formation from grouping of parameters extracted from processed event objects 130. In one embodiment, an encounter object may be formed at least in part from processing of one or more event objects 130. For example, after event objects 130 are received and initially processed by event processing and integration layer 140, parameters parsed from the processed event objects 130 may be associated and/or grouped to form one or more encounter objects to express attributes of corresponding patient encounters. For example, parameters parsed event objects 130 of a particular type of clinical event involving a particular patient and a particular service provider at a particular time window may be grouped to characterize a particular patient encounter to be expressed in an encounter object.

In some embodiments, the encounter grouping engine 154 may apply one or more configurable parameters 152, which may be stored in a memory, to correlate and/or associate parameters from different received encounter objects. For example, a configurable parameter 152 may define a rule to group all clinical events expressed by a corresponding multiple encounter objects occurring within a specific 24-hour period or a specific month period for a single patient into a single encounter group for facility billing purposes. Grouping may also combine parameters of encounter objects expressing multiple distinct encounters that occur over a defined period of time but are intended to be billed together. For example, multiple infusion services delivered to a patient over the course of a month expressed by corresponding encounter objects may be grouped into a single encounter group for billing purposes. Also, in some scenarios, it may be desirable to bundle charges preceding an inpatient encounter with charges during the inpatient encounter. Here, clinical services provided prior to an inpatient admission expressed as encounter objects may be associated with an encounter group corresponding to the provided clinical services preceding and during the inpatient encounter for consolidated billing or reporting. This process may directly correspond to the recited process of combining parameters of a plurality of encounter objects into a single encounter group, from which one or more claims can be created. This aggregation of related but separate encounter objects into a cohesive group may enable accurate and comprehensive claim generation. In an implementation, attributes of an encounter group may be expressed in one or more electronic documents stored as signals and/or states in a non-transitory memory. Such attributes may include, for example, an expression of parameters from which one or more claims may be created for electronic submission to a paying party (e.g., by application of processes by one or more applications 180 as discussed herein).

In some embodiments, events identified in event objects 130 may include a patient's interaction with one or more medical service providers. A single patient visit may be a complex multi-encounter event that unfolds over time. Each encounter expressed in an encounter object may itself be composed of numerous smaller events, such as patient check-in, the recording of vital signs, the performance of a procedure, the entry of a diagnosis and/or patient check-out, just to provide a few examples. In an embodiment, each of these discrete occurrences may be captured and transmitted to a healthcare information system implemented in server computing device 120 as one or more distinct event objects 130 to be mapped to an encounter object.

In an embodiment, parameters parsed from event objects 130 indicative of patient encounters may be mapped to an encounter object in a more structured data format. This mapping process may involve parsing the parameters from raw event objects 130, validating parsed parameters, and associating validated parameters with a specific patient and a specific, logical patient encounter. For example, multiple event objects 130 generated during a single office visit may be processed and mapped by server computing device 120 to multiple encounter objects to be combined in a single, unified encounter group object that represents an entirety of that visit (e.g., multiple patient encounters in the same visit).

Furthermore, multiple patient encounters may be aggregated into logical groups for more efficient processing. In some embodiments, multiple patient encounters, which may be identified in a plurality of distinct encounter objects, may be combined in and/or mapped to a single, logical encounter group. This grouping may be performed based, at least in part, on a set of predefined or configurable criteria. Such criteria may include, for example, temporal attributes, such as grouping multiple (e.g., all) patient encounters that occur within a single hospital admission or a 24-hour period. Criteria for grouping encounter objects to an encounter group may also be based on encounter types, such as grouping a series of related outpatient visits for a specific diagnosis or treatment plan into a single episode of care. Rules for combining encounters may also be configured by individual health systems, or may be configured automatically based on payer billing rules. Users may also manually combine encounters into a single encounter group. Such an encounter group may then serve as a comprehensive data package for subsequent processing, such as the generation of a single, consolidated facility claim or for clinical review.

An output of encounter grouping engine 154 may then be passed to a payer compliance check module 156. In some embodiments, payer compliance check 156 may apply a set of payer-specific rules to the encounter group to ensure that activities adhere to requirements of a particular paying party. For instance, payer compliance check 156 may verify that necessary documentation and/or authorizations associated with grouped encounters are present. The payer compliance check 156 may then provide validated and structured items to an API to initiate one or more calls to applications 180 to further formulate a claim for electronic submission to that paying party, such as an insurer.

A final claim may be prepared and managed by a claim submission workflow 189, which may be a component of applications 180. The claim submission workflow 189 may incorporate aspects and/or parameters from the patient graph 160 for a particular affected patient. For example, before submitting a claim, claim submission workflow 189 may query patient graph 160 to retrieve the patient's current insurance policy number, demographic information and/or active deductibles. This may enable a submission of a complete and accurate final claim to a paying party that is based not only on parameters parsed from recent event objects 130, but also the most current contextual information maintained in patient graph 160. Parameters from one or more event objects 130 expressing an encounter may be mapped to multiple claims as a single encounter group, which may result in electronic submission of distinct claims for different paying parties or different claim types (e.g., professional and facility claims).

The combination of encounter grouping engine 154, payer compliance check 156 and use of configurable parameters 152 as shown in FIG. 9 provides a specific improvement in computing technology by creating an automated data correlation and pre-validation engine. This may address the technical problem of operational inefficiencies caused by the need to manually review and aggregate disparate event data from fragmented systems to form a billable unit. For example, encounter grouping engine 154 may be configured to apply configurable parameters 152 to automatically correlate and associate parameters from separate, asynchronous event objects 130 expressing multiple patient encounters into a single, logically coherent encounter group. This may transform a computer from a passive storage system into an active data synthesizer capable of performing a complex logical aggregation task previously reliant on human cognition. Subsequent processing by the payer compliance check 156 may further enhance the computer's function by automatically pre-validating this newly created data structure against a set of payer-specific rules before it enters the final claim submission workflow 189. Integration of encounter grouping engine 154, payer compliance check 156 and use of configurable parameters 152 may operate as a more efficient and reliable computing tool, one that is specifically adapted to automate the pre-processing and validation of complex healthcare data, thereby overcoming the inefficiencies of manual cross-referencing and data reconciliation.

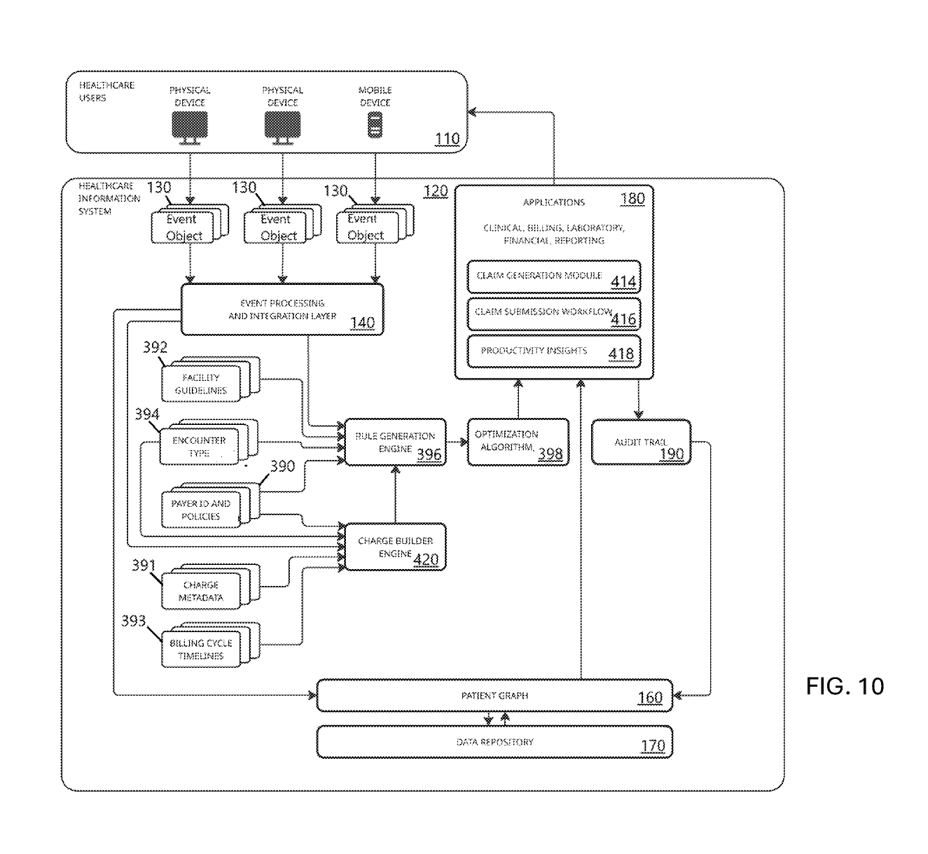

FIG. 10 is a block diagram illustrating an architecture for charge creation within a healthcare information system implemented on server computing device 120, according to some embodiments. In an implementation, encounter groups (such as those formed by the encounter grouping engine 154 (as shown in FIG. 9), may be further processed to construct compliant and optimized claims for electronic submission to a paying party.

In one embodiment, a rule generation engine 396 may process encounter groups by applying parameters from a plurality of contextual parameter sources. Rule generation engine 396 may apply facility guidelines 392 to incorporate any facility-specific billing policies or charge master rules. Rule generation engine 396 may also apply encounter type parameters 394, which can be used for associating the encounter group with a particular encounter type according to treatment codes (e.g., CPT codes) and/or diagnosis codes (e.g., ICD-10 codes). In an example, services rendered by an emergency department and impatient services may be associated with different encounter types, for example. Rule generation engine 396 may synthesize these inputs along with payer policies 390 and parameters parsed from original source event objects 130 to generate a dynamic set of rules that govern how a claim should be constructed for that specific encounter and/or encounter group (e.g., in downstream processing at applications 180), thereby initiating the creation of a claim for electronic submission to a paying party.

In some embodiments, a charge builder engine 420 may apply to parameters parsed from event objects 130 charge metadata 391, which may include standard rates and service codes, and billing cycle timelines 393. Charge builder engine 420 may generate specific charge items to be incorporated into a claim. In one embodiment, charge builder engine 420 may identify charges to be applied to a particular encounter group, and quantify particular amounts to be applied to the identified charges. Both the rule generation engine 396 and the charge builder engine 420 may also query patient graph 160 to ensure the rules and charges are contextually appropriate for the specific patient.

Optimization algorithm 398 may further process charges from charge builder engine 420 and rules from rule generation engine 396. In some embodiments, the optimization algorithm 398 may analyze and refine descriptive parameters of an encounter group to ensure correctness and completeness, and to conform the parameters of the encounter group to a format suitable for an API. This may allow structured data to be seamlessly consumed by downstream applications.

Conformed data received at an API may be passed to a claim generation module 414, which may be part of applications 180. Claim generation module 414 may also apply productivity insights 418 to further refine a claim for electronic submission or to provide feedback to users. For example, a productivity insight may flag a claim as potentially under-coded or missing a common associated charge. Finally, claim generation module 414 may generate a final claim for electronic submission to a paying party according to a defined claim submission workflow 416. This claim generation process for a particular claim may be recorded in audit trail 190.

The particular combination of rule generation engine 396, optimization algorithm 398, charge builder 420, and claim generation module 414 as shown in FIG. 10 may provide a specific improvement in computing technology by creating a dynamic, context-aware financial transaction engine. This addresses the technical problem of operational inefficiencies that arise in some computing systems in healthcare that are unable to autonomously synthesize complex, disparate, and frequently changing rule sets to create compliant financial claims. For example, rule generation engine 396 may receive and process multiple, independent data sources-such as payer policies 390, facility guidelines 392, and encounter type parameters 394-and dynamically generate a specific, context-sensitive set of rules for a given encounter and/or encounter group. This may transform a computer from a passive storage device that requires a human to look up and apply rules into an active system that can synthesize these rules itself. This dynamically generated rule set may then be used to automatically construct charge data from clinical event parameters. Optimization algorithm 398 may further refine this process, and resulting productivity insights 418 may provide a feedback mechanism that improves a system's function over time. Integration of rule generation engine 396, optimization algorithm 398, charge builder 420, and claim generation module 414 may enable a more efficient and intelligent computing tool that is specifically adapted to automate a complex, logic-based task of claim construction without the need for manual cross-referencing and intervention, thereby overcoming the fragmentation inherent in healthcare transaction processing systems.

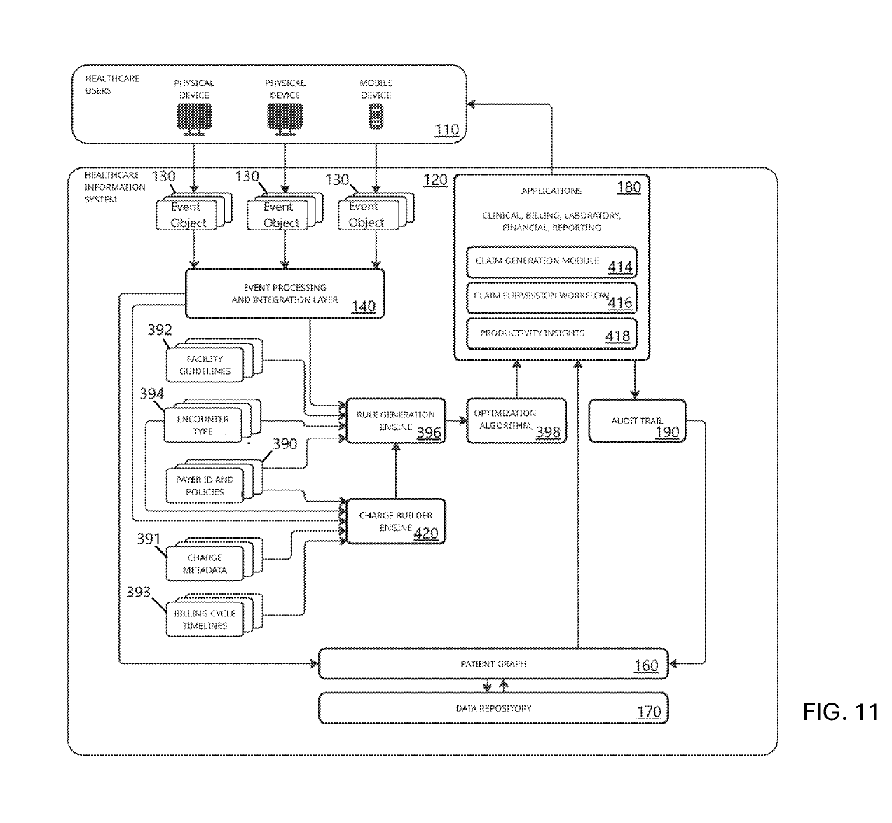

FIG. 11 is a block diagram illustrating an architecture for multi-format or multi-path claim generation within a healthcare information system implemented in server computing device 120, according to some embodiments. In the field of medical billing, different types of claims may be generated using different claim forms and submission workflows for different paying parties and/or different types of medical encounters. For example, in a single patient encounter, such as a surgical procedure performed in a hospital, at least two distinct claims may be formed: a professional claim for the surgeon's services (e.g., submitted on a CMS-1500 form) and a facility claim for the use of the hospital's operating room, staff, and supplies (e.g., submitted on a UB-04 form). In one embodiment, the system shown in FIG. 11 may handle such complexity by providing multiple, selectable workflows and/or claim forms.

In medical billing, multiple different paying parties may settle portions of a patient encounter or encounter group. For example, a portion of an encounter group may be settled via electronic submission of a first claim to a primary paying party, and electronic submission of at least a second claim to one or more additional paying parties (e.g., secondary paying party and/or tertiary paying party). In another embodiment, the system shown in FIG. 11 may be capable of changing a claim form type after a claim is submitted to a primary paying party but before it is sent to a secondary paying party. This may, for example, enable submission of a single claim to two different paying parties in different formats. This may be extended to tertiary paying parties and beyond.

In some embodiments, an API may direct elements of a claim, which have been constructed from an encounter group and processed by the rule generation engine 396 and charge builder engine 420, to one of a plurality of claim generation modules. This routing decision may be based on parameters within the processed elements of the claim, such as encounter type 394, payer ID and policies 390, the provider type, the provider location, and/or rules derived from payer policies 390, just to provide a few examples.

As shown in FIG. 11, an API may direct parameters of a claim to be processed by a first claim generation module, claim A generation module 424, according to a first claim submission workflow, claim A submission workflow 426. Following the example above, this path may generate and electronically submit a professional claim on a CMS-1500 form, for example. Additionally, for the same patient encounter, the API may direct associated facility charges to be processed by a second claim generation module, claim B generation module 434, according to a second and distinct claim submission workflow, claim B submission workflow 436. This second path may generate and electronically submit a facility claim on a UB-04 form, for example. While this example describes two pathways, the architecture of FIG. 11 may support any number of different claim generation modules and workflows as desired.

The system of FIG. 11 may further apply productivity insights 418 to enhance efficiency and accuracy of each workflow. Actions taken within the claim A generation module 424 and the claim B generation module 434, as well as their respective submission workflows, may be recorded in audit trail 190, enabling a complete and traceable history for all claims generated from a group of patient encounters expressed as an encounter group.

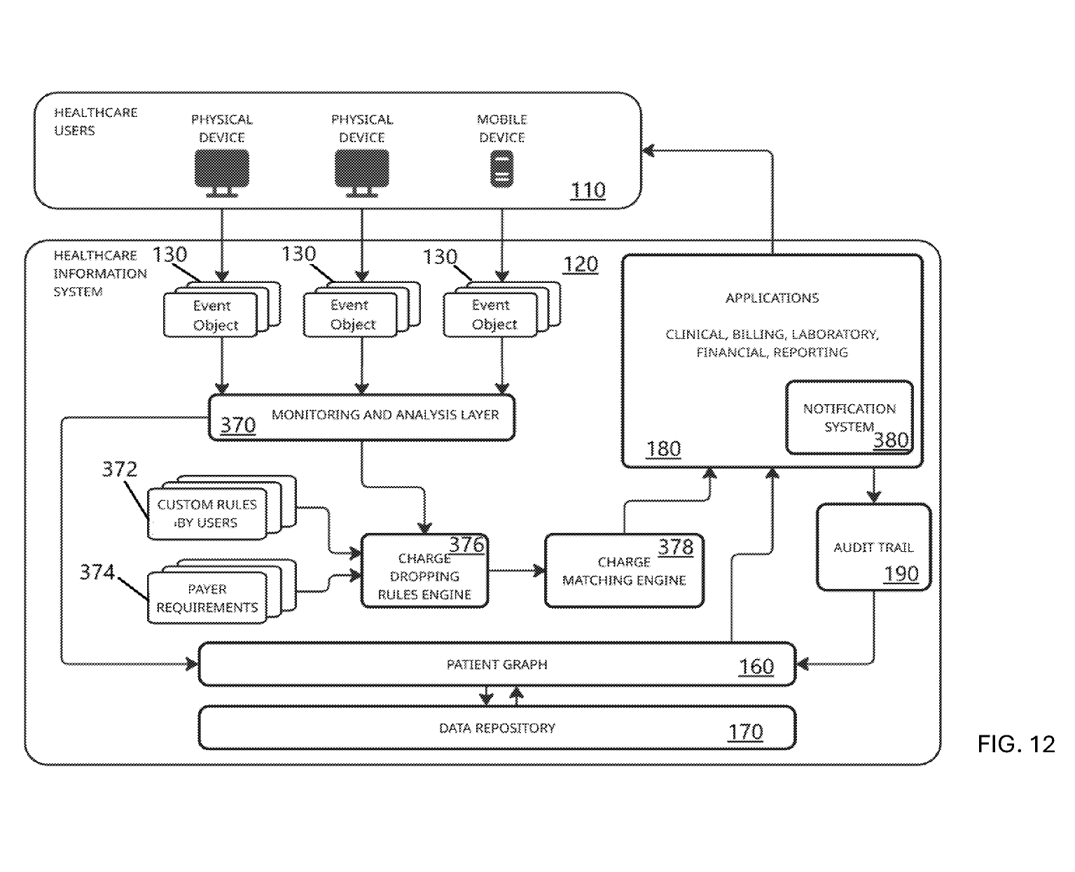

The combination of the claim A generation module 424 and claim B generation module 434, each with its respective submission workflow (426, 436) (e.g., along with the charge dropping rules engine 376 and charge matching engine 378 shown in FIG. 12), may provide a specific improvement in computing technology by creating an integrated claim management system that overcomes the operational inefficiencies of processing complex healthcare encounters. For example, this architecture may address the technical challenge where a single set of clinical event data must be bifurcated to generate multiple, distinct claim types-such as a professional claim and a facility claim-each with its own submission rules and financial lifecycle, instead of being handled by separate, fragmented systems. The system may intelligently route processed charges to an appropriate generation module (424 or 434), enabling concurrent creation of different claim forms from a single, unified data source. This may transform the computer from a series of siloed "form-filling" tools into a cohesive transaction coordinator that understands the interrelationship between different claims arising from a single encounter. By integrating parallel claim generation with unified integrity management, the system may function as a more efficient, reliable, and automated computing tool specifically adapted for the complexities of healthcare finance.

FIG. 12 is a block diagram illustrating an architecture for charge integrity management and modification within a healthcare information system implemented on server computing device 120, according to some embodiments. Charges may be automatically generated and posted based on, for example, patient admission or transfer events, and order additions, signatures, or cancellations. In the lifecycle of a medical claim, it is common for circumstances to change or for new information to become available after initial charges for a claim have been generated. For example, a procedure may be canceled, a diagnosis may be revised, and/or a paying party may issue a new policy. Such changes may initiate a dropping of specific charges to a pending claim or a yet-to-be-drafted future claim (or dropping of specific charges at the encounter level to be later included on a claim). The system shown in FIG. 12 may manage this dynamic process in an automated and auditable manner.

Monitoring and analysis layer 370 (e.g., incorporated within or working in conjunction with event processing and integration layer 140) may monitor and detect, in parameters parsed from one or more newly received event objects 130, information that is suggestive of a change to a pending claim, such as inclusion or removal of a charge on the pending claim. For instance, an event object 130 indicating a "procedure canceled" status or a revised diagnosis code may be detected by monitoring and analysis layer 370 as a trigger for a potential charge modification.

Upon detection of such a trigger event by monitoring and analysis layer 370, charge dropping rules engine 376 may apply a set of rules to determine whether a charge should be added to or removed from a pending claim. These rules may be derived from multiple sources including, for example, custom rules by users 372 (e.g., a facility's internal policy to not bill for certain canceled services) and/or payer requirements 374 (e.g., a payer's specific policy on bundling services or not covering certain procedures under new circumstances). Charge dropping rules engine 376 may evaluate whether the triggering event against these rules is to make a definitive determination on whether a charge is to be included or removed from a pending claim.

If charge dropping rules engine 376 determines that a charge is to be included or removed, charge matching engine 378 may associate a triggering event with a specific, previously generated charge and/or locate the specific pending claim to which the charge is to be included or removed. Charge matching engine 378 may do this by using identifiers from the event object 130, such as a patient ID, encounter ID, and date of service, to query the data repository 170 or patient graph 160, and pinpoint correct charge to be included or removed and its associated claim.

Once a charge has been identified and marked for inclusion or removal, a notification system 380 may initiate messages to update system records. Specifically, notification system 380 may initiate messages to audit trail 190 to create an immutable record that accounts for the included or removed charge, including the reason and timestamp for the action. Concurrently, the patient graph 160 may be updated accordingly. For example, a state of the charge within the patient graph 160 may be changed to "included" or "voided," ensuring that the patient's unified record remains accurate and reflects the most current state of the billing process.

The combination of the charge dropping rules engine 376, charge matching engine 378, and notification system 380, operating in conjunction with the patient graph 160 as shown in FIG. 12, provides a specific improvement in computing technology by creating an automated mechanism for maintaining charge integrity for claims to be electronically submitted for payment. This addresses the technical problem of operational inefficiencies caused by fragmented and complex data processing where charge postings and modifications are typically handled through slow, error-prone manual intervention across separate systems. Specifically, the charge dropping rules engine 376 is configured to automatically apply a complex set of payer requirements 374 and custom user rules 372 to new event data, making a real-time determination on the validity of an existing charge or whether a charge is to be included. Upon this determination, charge matching engine 378 may execute a targeted query to patient graph 160 to efficiently and accurately locate the specific charge to be modified. This may transform a computer system from a passive repository of financial data into an active, self-auditing system capable of autonomously tracking charges and resolving data conflicts. A subsequent update to patient graph 160 and alert from notification system 380 may create a cohesive, closed-loop process that operates entirely within a computing system to enable data consistency without requiring manual cross-system reconciliation, thereby representing a more efficient and reliable computing method for managing healthcare transactions.

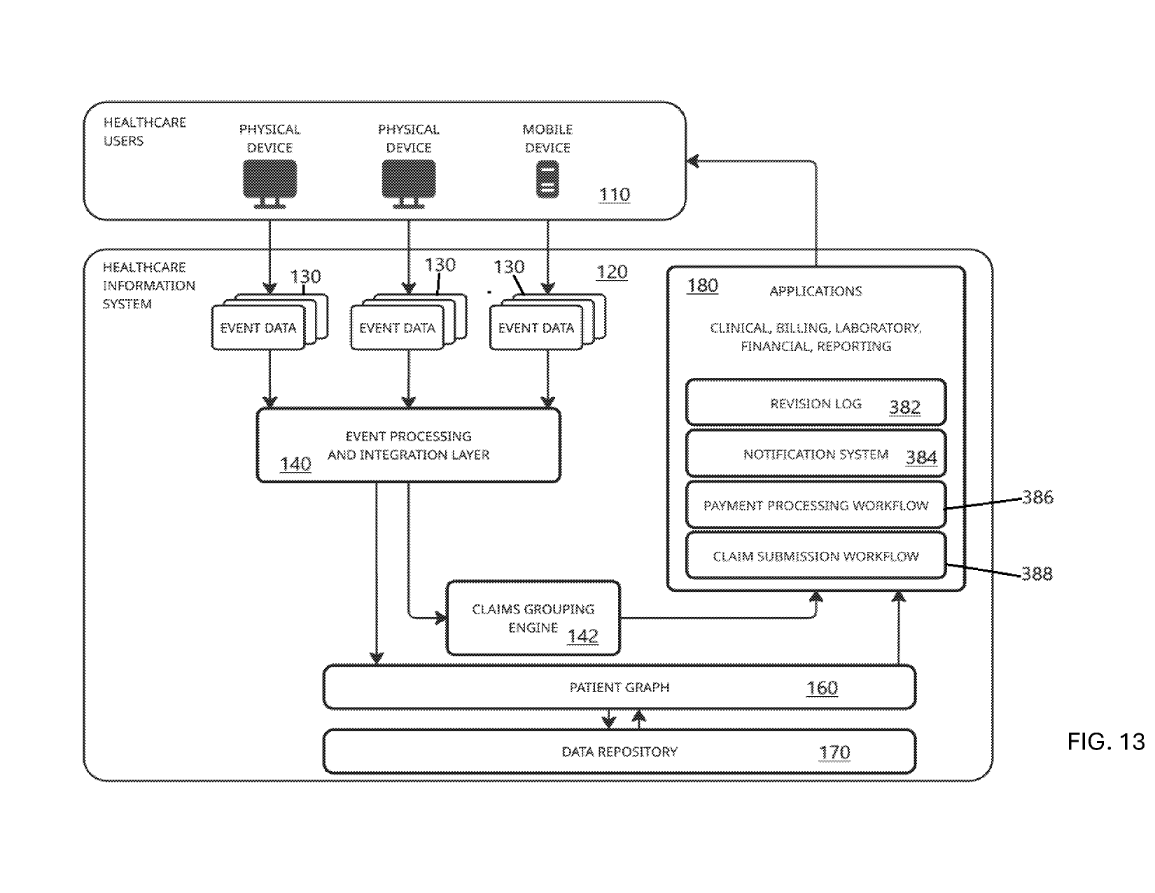

FIG. 13 is a block diagram illustrating an architecture for managing the financial lifecycle of a medical claim, according to some embodiments. In some embodiments, applications 180 may manage the life of a claim from an initial generation of charges, which may arise from patient encounters, through to final payment and/or settlement. Throughout this lifecycle, a claim may be associated with any one of several states. These states are associated with and/or tied to a particular claim submission workflow 388 and/or a payment processing workflow 386, which are managed within the applications 180. The state of a claim may indicate, for example, a current payment account balance, whether the claim has been submitted, has been revised, is past due, has been partially paid, or includes an amount in dispute.

The system of FIG. 13 may dynamically update a state of a claim in response to new information. In some embodiments, a claims grouping engine 142 may process and/or respond to parameters parsed from new event objects 130 to initiate changes to an existing claim's state. In a particular implementation, claims grouping engine 142 may group claim revisions, transactions, and related data automatically into a single claim account. In another example, an event object 130 originating from a payer's system may contain data indicating that a partial payment has been made. Claims grouping engine 142 may process this event and initiate an update to the corresponding claim. Similarly, an event object 130 from a patient's mobile device via a client computing device 110 may indicate a dispute over a specific charge. In response, claims grouping engine 142 may initiate a change to the claim's state to "partially paid" or "in dispute," respectively, and may trigger the appropriate sub-process within the payment processing workflow 386 or a notification via the notification system 384.

To ensure a complete and auditable history of every claim, any resulting changes to a claim may be recorded in a revision log 382. Revision log 382 may be configured to reflect each change to a state of a claim. For instance, revision log 382 may record a timestamp of the change, the party or system that initiated the change (e.g., an automated payment event), the previous state, and the new state. Revision log 382, which may be a component of or linked to the audit trail 190, may thereby provide a transparent and immutable record of the entire financial history of the claim from its generation to its final resolution. All such changes are also reflected in the patient graph 160 to maintain a single source of truth.

The combination of features shown in FIG. 13 may provide a specific improvement in computing technology that addresses the technical problem of fragmented and inefficient post-submission claim processing. Specifically, the claims grouping engine 142 may function as an intelligent event router that is configured to receive and process asynchronous event objects 130, such as payment notifications or dispute flags, and correlate them to a specific, existing claim stored within the system. Upon successful correlation, claims grouping engine 142 may initiate an update to a claim's state by triggering the appropriate workflow, such as the payment processing workflow 386. This state change-for instance, from 'submitted' to 'partially paid'-may be immutably recorded in the revision log 382, creating an auditable history of the claim's financial lifecycle. Simultaneously, notification system 384 may be triggered to automatically alert relevant stakeholders of the change. This architecture may transform a claim from a static, submitted document into a dynamic, stateful data object within a computing system itself. By creating a cohesive, closed-loop system for automatically processing relevant events and maintaining a complete, auditable history in revision log 382, this specific arrangement may overcome the reliance on disconnected reconciliation systems and manual tracking, thereby improving the computer's ability to function as a more reliable and efficient financial transaction management tool.

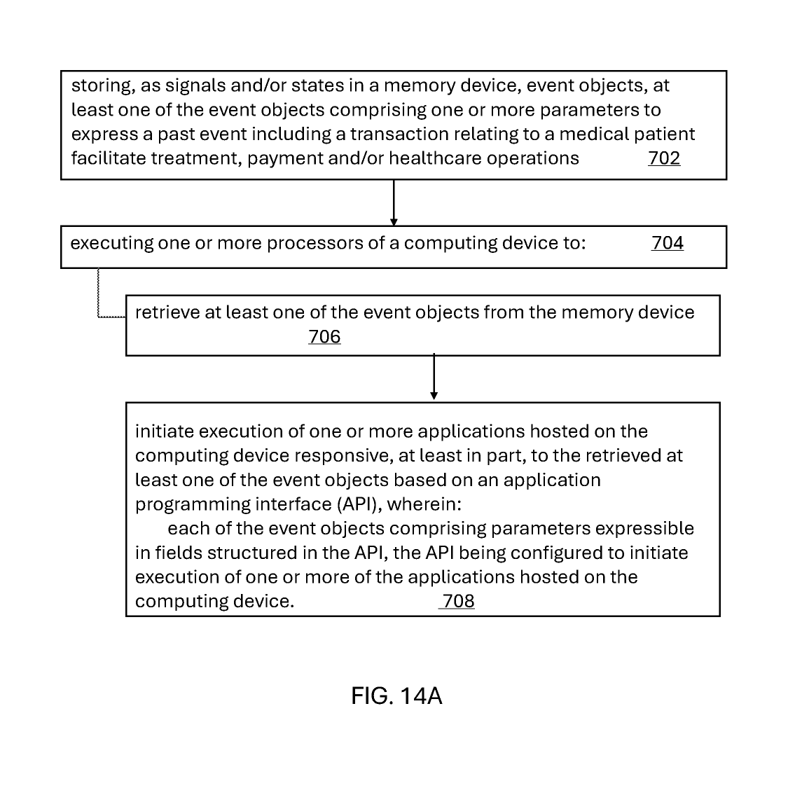

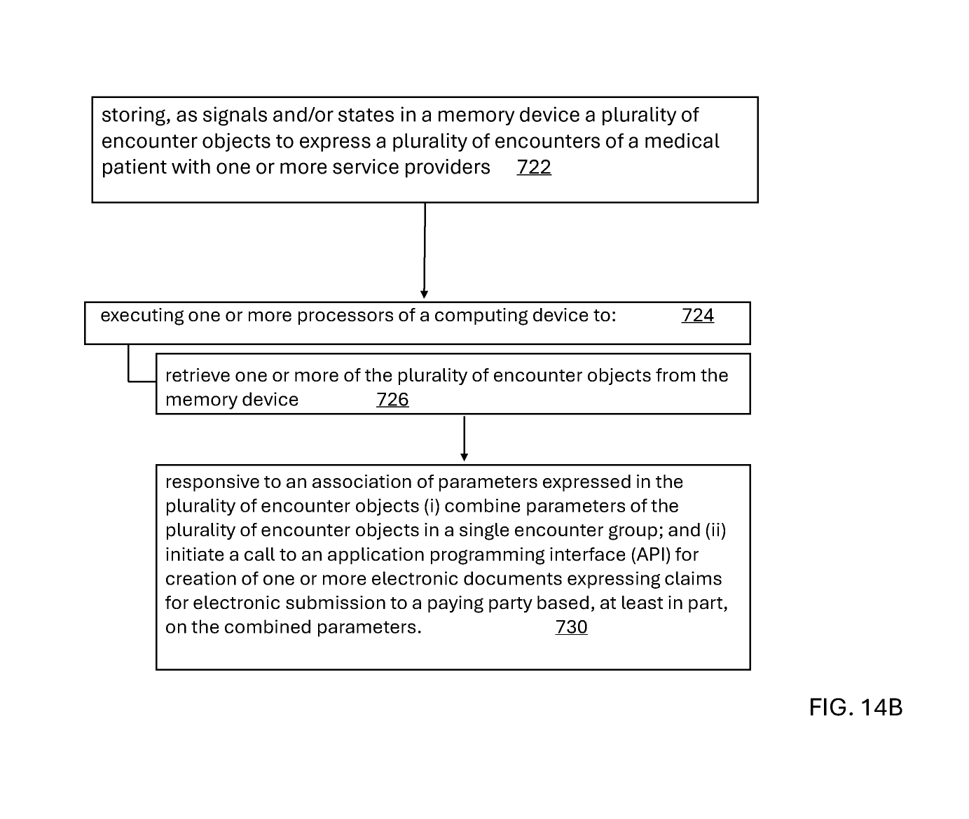

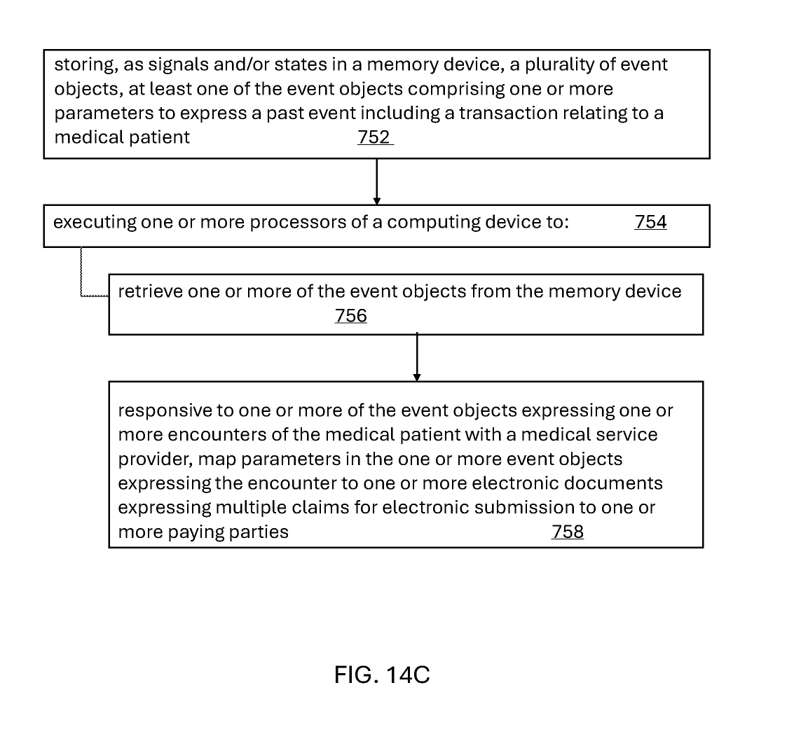

FIG. 14A is a flowchart illustrating an exemplary technique for processing healthcare transactions, according to some embodiments. Actions described in FIG. 14A may be performed by the features and elements of an implementation of a healthcare information system on server computer device 120 shown in and discussed in connection with FIG. 1. Block 702 comprises storing, as signals and/or states in a memory device, a plurality of event objects. This action corresponds to the server computing device 120 of FIG. 1 receiving one or more event objects 130 from client computing devices 110 and storing them in a memory and/or a temporary buffer. An event object 130 comprises one or more parameters expressing a past event, such as a transaction relating to a medical patient (e.g., administrative transactions or clinical encounter with service provider).

Block 704 comprises executing one or more processors of a computing device. This may correspond to the execution of instructions by the processor(s) of the server computing device 120 to perform the subsequent operations to process transactions. At block 706, the processor(s) may retrieve at least one of the event objects 130 from the memory device. This action may be performed by the event processing and integration layer 140 shown in FIG. 1, which may retrieve stored event objects 130 from the data repository 170 for processing. At block 708, the processor(s) initiate the execution of one or more applications responsive, at least in part, to the retrieved event object 130. This action may be performed by event processing and integration layer 140, which, after processing a retrieved event object 130, may initiate a call to an API to initiate a function within the applications 180. Specific application or workflow initiated may be determined by the parameters within the processed event object 130.

In one embodiment, parameters of an event object 130 retrieved and processed at block 706 may be used to update a state of patient graph 160 so that patient graph 160 reflects most current information, such as adding a new diagnosis or a new procedure to the patient's record. Subsequently, action of initiating the execution of one or more applications 180 may not be based solely on the data within triggering event object(s) 130. For example, an application 180 may also use parameters maintained in patient graph 160 to provide additional context for the execution of the application. This context may include, for example, a patient's insurance status, existing comorbidities, or progress within a pre-defined care plan, just to provide a few examples.

FIG. 14B is a flowchart illustrating an exemplary process for generating one or more claims from a plurality of encounter objects, according to some embodiments. The actions described in the flowchart may be performed by the features and elements of a healthcare information system implemented on server computing device 120 shown in and discussed in connection with FIGS. 9 and 10, for example. Block 722 may store in a memory device encounter objects expressing a plurality of encounters of a medical patient with one or more service providers. At block 724, one or more processors of a computing device may execute subsequent operations.

At block 726, the processors retrieve two or more of the stored encounter objects from the memory device. As pointed out above, encounter objects stored in a memory and retrieved at block 726 may be obtained from an external source or from an analysis of processed event objects 130.

Block 730 comprises a two-part action that is responsive to an association of parameters expressed in the plurality of retrieved encounter objects. First, at (i), block 730 comprises combining parameters of the plurality of encounter objects into a single encounter group. This action is performed by the encounter grouping engine 154 shown in FIG. 9. Encounter grouping engine 154 may apply configurable parameters 152 to correlate and associate parameters from the different encounter objects, thereby aggregating them into a single, logical encounter group for billing purposes, for example.

Second, at (ii), block 730 may create one or more claims for electronic submission to a paying party based, at least in part, on the combined parameters of the single encounter group formed in (ii). This action is performed by the combination of engines detailed in FIG. 10. Specifically, rule generation engine 396 may process an encounter group by applying rules derived from facility guidelines 392 and encounter type parameters 394. Concurrently, charge builder engine 420 may apply charge metadata 391 and billing cycle timelines 393 to construct the specific financial charges for the encounter group. The output of these engines is then used by the claim generation module 414 (FIG. 10) to create the final claim, which is then managed by the claim submission workflow 189 (FIG. 9) for electronic submission.

FIG. 14C is a flowchart illustrating an exemplary process for generation of multiple claims from one or more patient encounters, according to some embodiments. The actions described in the flowchart may be performed by the features and elements of the healthcare information system 120 shown in and discussed in connection with FIGS. 9 and 10. Block 752 comprises storing a plurality of event objects 130 related to a medical patient in a memory device. Block 754 comprises execution of one or more processors of a computing device to perform the subsequent operations.

Block 756 may comprise retrieving two or more of the stored event objects 130 from the memory device. This action may be performed by event processing and integration layer 140 (FIG. 9), which retrieves relevant event objects 130 for processing and aggregation. Responsive to one or more event objects expressing one or more encounters of the medical patient with a medical service provider, block 758 may map parameters in event objects 130 to multiple claims for electronic submission to a paying party. This mapping may be performed by a combination of the features shown in FIGS. 9, 10 and 11. In an embodiment, retrieved event objects 130 may be parsed and contextually analyzed so that parameters parsed from the retrieved event objects 130 are mapped to one or more encounter objects to express a corresponding one or more patient encounters with a service provider. As pointed out above, encounter grouping engine 154 (FIG. 9) may map parameters of multiple encounter objects into a single encounter group. The rule generation engine 396 (FIG. 11) may then process this single encounter group. By applying rules derived from payer policies 390 or encounter type parameters 394, rule generation engine 396 may determine that the charges associated with the encounter group be split into different categories. For example, it may determine that one set of charges corresponds to professional services while another corresponds to facility services.

Based on this determination, the charge builder engine 420 (FIG. 10) generates the distinct sets of charges. The claim generation module 414 (FIG. 10) may then receive these categorized charges and "maps" them by creating multiple, separate claims-one for the professional charges and another for the facility charges. Each of these distinct claims may then be managed by claim submission workflow 189 (FIG. 9) for electronic submission to the appropriate one or more paying parties.

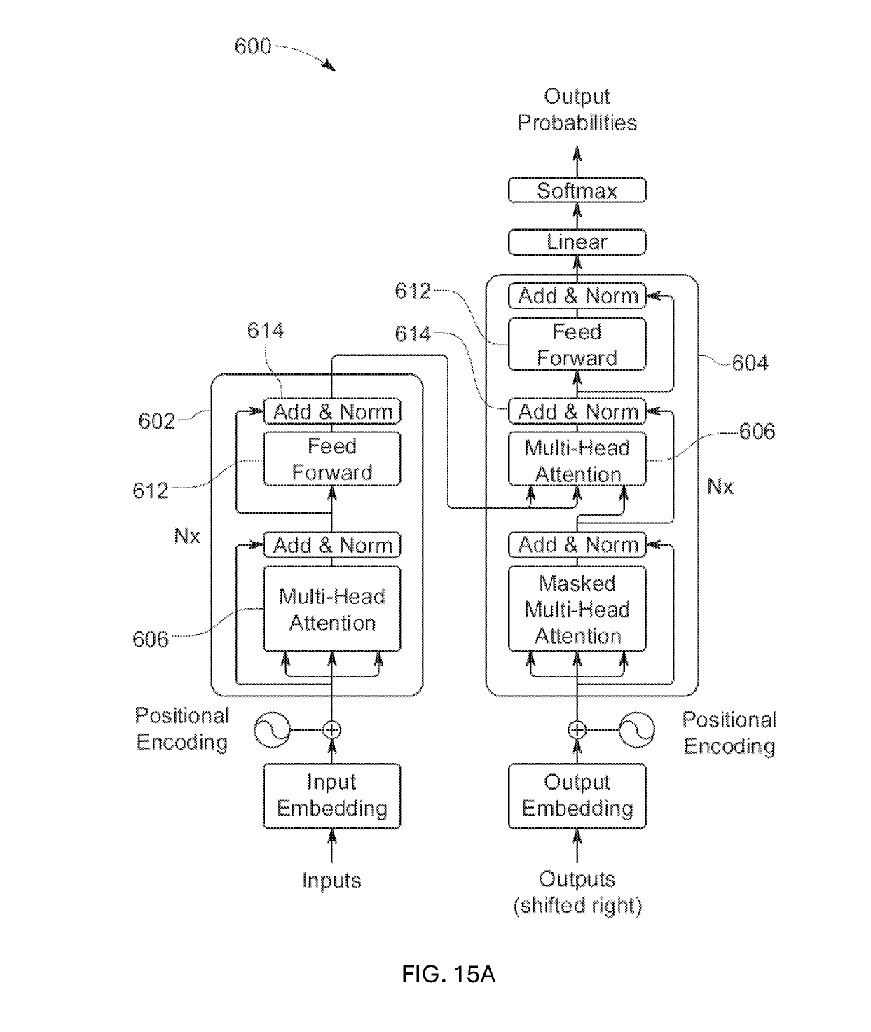

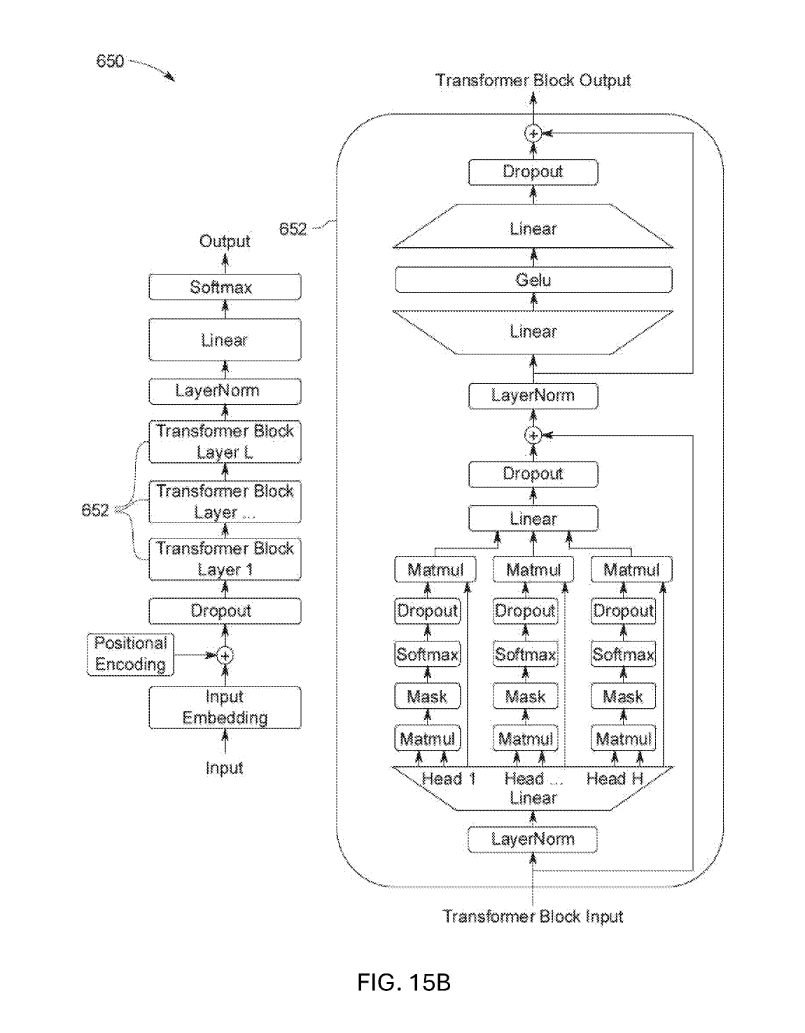

According to an embodiment, one or more aspects of the present disclosure may be implemented, at least in part, as a pretrained large language model (LLM) to perform natural language processing (NLP) and logical reasoning. For example, portions of semantic extraction 212 (FIG. 2), contextual accuracy model 214 (FIG. 2), event monitoring engine 355 (FIG. 3), sentinel execution 357 (FIG. 3), semantic data processing 304 (FIG. 4), language processing engine 272 (FIG. 8), contextual analysis model 274 (FIG. 8) may be configured to employ pretrained models employing one or more of a neural network model, probabilistic model, reinforcement learning model, tree-based model, Markov tree model, deep learning model or large language models, just to provide a few examples. In an embodiment, such a model may comprise a pretrained LLM that is trained from a corpus of medical documents (e.g., clinical notes, diagnoses, treatments, billing transactions, etc.). FIG. 15A is a schematic diagram of one embodiment of a neural network implementation of a pretrained model, for example. FIG. 15B is a schematic diagram of another embodiment of a neural network implementation of a pretrained LLM model using a series of transformers 652. In one implementation, inputs for neural network model 600 and/or 650 may comprise a series of words that are preprocessed (e.g., converted to numbers or other input vectors) and provided in sequence to generate output probabilities of a subsequent word. Such a series of words may be obtained and/or parsed from clinical records of patient encounters from a large corpus of clinical records in a pretraining operation. Once the subsequent word is determined, the subsequent word may be combined with the input so that the next subsequent word may be determined, causing transformers to repeatedly predict the next word in a response to a prompt. In one implementation, an input sequence may be fixed at some value, such as 2048 words, and extra positions at the beginning may be padded with zeros. An output may similarly comprise an array of possible outcomes with associated probabilities, such that the most probable subsequent word may be selected as the next word in the response or output.

Because input vectors in this particular example may indicate only a single word and comprise many more zeros than ones, the input vectors may be embedded or encoded into a smaller multidimensional space at an input embedding element. In generative neural network model 600 in particular, the position of each resulting token in a sequence of inputs may be encoded and provided to a multi-head attention element 606 operable to predict a degree to which an input token is likely to impact an output. Feed-forward blocks 612 may each comprise a multi-layer neural network, operable to learn over time to predict the next word in a sequence. An add & norm block 614 may combine and normalize outputs of multiple previous blocks.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms "coupled" and "connected" are used in a manner so that the terms are not synonymous. Similar terms may also be used in a manner in which a similar intention is exhibited. Thus, "connected" is used to indicate that two or more tangible components and/or the like, for example, are tangibly in direct physical contact. Thus, using the previous example, two tangible components that are electrically connected are physically connected via a tangible electrical connection, as previously discussed. However, "coupled," is used to mean that potentially two or more tangible components are tangibly in direct physical contact. Nonetheless, "coupled" is also used to mean that two or more tangible components and/or the like are not necessarily tangibly in direct physical contact, but are able to co-operate, liaise, and/or interact, such as, for example, by being "optically coupled." Likewise, the term "coupled" is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Unless otherwise indicated, in the context of the present patent application, the term "or" if used to associate a list, such as A, B, or C, is intended to mean A, B, and C, here used in the inclusive sense, as well as A, B, or C, here used in the exclusive sense. With this understanding, "and" is used in the inclusive sense and intended to mean A, B, and C; whereas "and/or" can be used in an abundance of caution to make clear that all of the foregoing meanings are intended, although such usage is not required. In addition, the term "one or more" and/or similar terms is used to describe any feature, structure, characteristic, and/or the like in the singular, "and/or" is also used to describe a plurality and/or some other combination of features, structures, characteristics, and/or the like. Likewise, the term "based on" and/or similar terms are understood as not necessarily intending to convey an exhaustive list of factors, but to allow for existence of additional factors not necessarily expressly described.

The terms “correspond”, “reference”, “associate”, and/or similar terms relate to signals, signal samples and/or states, e.g., components of a signal measurement vector, which may be stored in memory and/or employed with operations to generate results, depending, at least in part, on the above-mentioned, signal samples and/or signal sample states. For example, a signal sample measurement vector may be stored in a memory location and further referenced wherein such a reference may be embodied and/or described as a stored relationship. A stored relationship may be employed by associating (e.g., relating) one or more memory addresses to one or more other memory addresses, for example, and may facilitate an operation, involving, at least in part, a combination of signal samples and/or states stored in memory, such as for processing by a processor and/or similar device, for example. Thus, in a particular context, “associating,” “referencing,” and/or “corresponding” may, for example, refer to an executable process of accessing memory contents of two or more memory locations, e.g., to facilitate execution of one or more operations among signal samples and/or states, wherein one or more results of the one or more operations may likewise be employed for additional processing, such as in other operations, or may be stored in the same or other memory locations, as may, for example, be directed by executable instructions. Furthermore, terms “fetching” and “reading” or “storing” and “writing” are to be understood as interchangeable terms for the respective operations, e.g., a result may be fetched (or read) from a memory location; likewise, a result may be stored in (or written to) a memory location.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples), for example, may be exchanged, such as between a server device and/or a client device, as well as other types of devices, including between wired and/or wireless devices coupled via a wired and/or wireless network, for example.

In the context of the present patent application, the term network device refers to any device capable of communicating via and/or as part of a network and may comprise a computing device. While network devices may be capable of communicating signals (e.g., signal packets and/or frames), such as via a wired and/or wireless network, they may also be capable of performing operations associated with a computing device, such as arithmetic and/or logic operations, processing and/or storing operations (e.g., storing signal samples), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as those that may employ differing architectures and/or that may be substantially compliant and/or substantially compatible with differing protocols, such as network computing and/or communications protocols (e.g., network protocols), may interoperate within a larger network.

The Internet refers to a decentralized global network of interoperable networks that comply with the Internet Protocol (IP). It is noted that there are several versions of the Internet Protocol. The term Internet Protocol, IP, and/or similar terms are intended to refer to any version, now known and/or to be later developed. The Internet includes local area networks (LANs), wide area networks (WANs), wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

The term electronic file and/or the term electronic document are used throughout this document to refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted that an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized to specify digital content and/or to specify a format thereof, such as in the form of an electronic file and/or an electronic document, such as a Web page, Web site, etc., for example. An Extensible Markup Language (“XML”) may also be utilized to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the terms “entry,” “electronic entry,” “document,” “object,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. In another embodiment, an electronic document, electronic content and/or digital content may comprise text, audio and/or image content formatted to be processed by a generative neural network model, or text, audio and/or image content generated by a generative neural network model. For one or more embodiments, an electronic document and/or an electronic file may comprise a Web page of code (e.g., computer instructions) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term parameters (e.g., one or more parameters) refer to material descriptive of a collection of signal samples, such as one or more electronic documents and/or electronic files, and exist in the form of physical signals and/or physical states, such as memory states. For example, one or more parameters, such as referring to an electronic document and/or an electronic file comprising an image, may include, as examples, time of day at which an image was captured, latitude and longitude of an image capture device, such as a camera, for example, etc. In another example, one or more parameters relevant to digital content, such as digital content comprising a technical article, as an example, may include one or more authors, for example. Claimed subject matter is intended to embrace meaningful, descriptive parameters in any format, so long as the one or more parameters comprise physical signals and/or states, which may include, as parameter examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the Internet Protocol (IP) may include IPv4, IPv6, and/or other later to be developed versions.

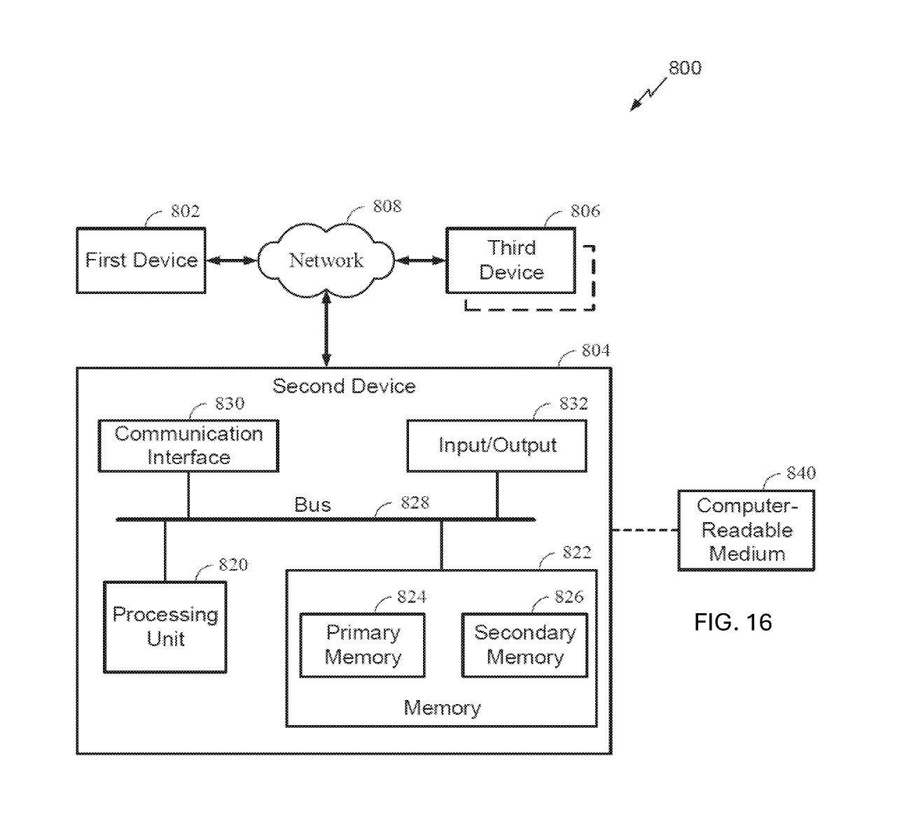

In one example embodiment, as shown in FIG. 16, a system embodiment may comprise a local network (e.g., device 804 and medium 840) and/or another type of network, such as a computing and/or communications network. For purposes of illustration, therefore, FIG. 16 shows an embodiment 800 of a system that may be employed to implement either type or both types of networks. Network 808 may comprise one or more network connections, links, processes, services, applications, and/or resources to facilitate and/or support communications, such as an exchange of communication signals, for example, between a computing device, such as 802, and another computing device, such as 806, which may, for example, comprise one or more client computing devices and/or one or more server computing devices. By way of example, but not limitation, network 808 may comprise wireless and/or wired communication links, telephone and/or telecommunications systems, Wi-Fi networks, Wi-MAX networks, the Internet, a local area network (LAN), a wide area network (WAN), or any combinations thereof.

Example devices in FIG. 16 may comprise features, for example, of a client computing device and/or a server computing device, in an embodiment. It is further noted that the term computing device, in general, whether employed as a client and/or as a server, or otherwise, refers at least to a processor and a memory connected by a communication bus. A "processor," for example, is understood to connote a specific structure such as a central processing unit (CPU) of a computing device which may include a control unit and an execution unit. In an aspect, a processor may comprise a device that fetches, interprets and executes instructions to process input signals to provide output signals. As such, in the context of the present patent application at least, computing device and/or processor are understood to refer to sufficient structure within the meaning of 35 USC § 112 (f) so that it is specifically intended that 35 USC § 112 (f) not be implicated by use of the term "computing device," "processor" and/or similar terms; however, if it is determined, for some reason not immediately apparent, that the foregoing understanding cannot stand and that 35 USC § 112 (f), therefore, necessarily is implicated by the use of the term "computing device," "processor" and/or similar terms, then, it is intended, pursuant to that statutory section, that corresponding structure, material and/or acts for performing one or more functions be understood and be interpreted to be described at least in FIGS. 1 - 13, 14A, 14B, 14C, 15A and 15B, and in the text associated with the foregoing figure(s) of the present patent application.

Referring now to FIG. 16, in an embodiment, first and third devices 802 and 806 may be capable of rendering a graphical user interface (GUI) (e.g., including a pointer device) for a network device and/or a computing device, for example, so that a user-operator may engage in system use. Computing device 804 may potentially serve a similar function in this illustration. Likewise, in FIG. 16, computing device 802 ('first device' in figure) may interface with computing device 804 ('second device' in figure), which may, for example, also comprise features of a client computing device and/or a server computing device, in an embodiment. Processor (e.g., processing device) 820 and memory 822, which may comprise primary memory 824 and secondary memory 826, may communicate by way of communication bus 828, for example. The term "computing device," in the context of the present patent application, refers to a system and/or a device, such as a computing apparatus, that includes a capability to process (e.g., perform computations) and/or store digital content, such as electronic files, electronic documents, measurements, text, images, video, audio, etc. in the form of signals and/or states. Thus, a computing device, in the context of the present patent application, may comprise hardware, software, firmware, or any combination thereof (other than software per se). Computing device 804, as depicted in FIG. 16, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, digital versatile disc (DVD) and/or other optical disc players and/or recorders, game consoles, satellite television receivers, cellular telephones, tablet devices, wearable devices, personal digital assistants, mobile audio and/or video playback and/or recording devices, Internet of Things (IOT) type devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, global positioning system (GPS) and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, global system for mobile communications (GSM), enhanced data rate for GSM evolution (EDGE), 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that is may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an Internet Protocol (IP) address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computer operating systems, such as Windows, iOS, Linux, a mobile operating system, such as iOS, Android, Windows Mobile, and/or the like. A computing device and/or network device may include and/or may execute a variety of possible applications, such as a client software application enabling communication with other devices. A computing and/or network device may also include executable computer instructions to process and/or communicate digital content. A computing and/or network device may also include executable computer instructions to perform a variety of possible tasks, such as browsing, searching, playing various forms of digital content, including locally stored and/or streamed video, and/or games . A computing and/or network device may also process input content as a prompt to one or more generative neural network models to provide output content. A computing and/or network device may also perform linguistic processing such as applying transforms to determine an embedding of tokens and/or apply attention models to determine service codes. The foregoing is provided merely to illustrate that claimed subject matter is intended to include a wide range of possible features and/or capabilities.

In FIG. 16, computing device 802 may provide one or more sources of executable computer instructions in the form of physical states and/or signals (e.g., stored in memory states), for example. Computing device 802 may communicate with computing device 804 by way of a network connection, such as via network 808, for example. As previously mentioned, a connection, while physical, may not necessarily be tangible. Although computing device 804 of FIG. 16 shows various tangible, physical components, claimed subject matter is not limited to computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 822 may comprise any non-transitory storage mechanism. Memory 822 may comprise, for example, primary memory 824 and secondary memory 826, additional memory circuits, mechanisms, or combinations thereof may be used. Memory 822 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 822 may be utilized to store a program of executable computer instructions. For example, processor 820 may fetch executable instructions from memory and proceed to interpret and execute the fetched instructions. Memory 822 may also comprise a memory controller for accessing device readable-medium 840 that may carry and/or make accessible digital content, which may include code, and/or instructions, for example, executable by processor 820 and/or some other device, such as a controller, as one example, capable of executing computer instructions, for example. Under direction of processor 820, a non-transitory memory, such as memory cells storing physical states (e.g., memory states), comprising, for example, a program of executable computer instructions, may be executed by processor 820 and able to generate signals to be communicated via a network, for example, as previously described. Generated signals may also be stored in memory, also previously suggested. In a particular implementation, processor 820 may include general processing cores and/or specialized co-processing cores (e.g., signal processors, graphical processing unit (GPU) and/or neural network processing unit (NPU)), for example.

Memory 822 may store electronic files and/or electronic documents, such as relating to one or more users, and may also comprise a computer-readable medium that may carry and/or make accessible content, including code and/or instructions, for example, executable by processor 820 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted that an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, service codes, tokens, computed likelihoods, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as "processing," "computing," "calculating," "determining", “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 16, processor 820 may comprise one or more circuits, such as digital circuits, to perform at least a portion of a computing procedure and/or process. By way of example, but not limitation, processor 820 may comprise one or more processors, such as controllers, microprocessors, microcontrollers, application specific integrated circuits, GPUs, NPUs, digital signal processors, programmable logic devices, field programmable gate arrays, the like, or any combination thereof. In various implementations and/or embodiments, processor 820 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 16 also illustrates device 804 as including a component 832 operable with input/output devices, for example, so that signals and/or states may be appropriately communicated between devices, such as device 804 and an input device and/or device 804 and an output device. A user may make use of an input device, such as a computer mouse, stylus, track ball, microphone, scanner, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A method, comprising:
storing, as signals and/or states in a memory device, event objects, at least one of the event objects comprising one or more parameters to express a past event including a transaction to facilitate treatment, payment and/or healthcare operations; and
executing one or more processors of a computing device to:
retrieve at least one of the event objects from the memory device; and
initiate execution of one or more applications hosted on the computing device responsive, at least in part, to the retrieved at least one of the event objects based on an application programming interface (API), wherein:
each of the event objects comprising parameters that are expressible in fields structured in the API, the API being configured to initiate one or more of the applications hosted on the computing device responsive to event objects.

2. The method of claim 1, wherein the one or more of the applications hosted on the computing device comprise at least one clinical medical application and at least one administrative medical application.

3. The method of claim 1, and further comprising further executing the one or more processors of the computing device to:
parse parameters from the retrieved at least one of the event objects; and
initiate a call to the one or more applications hosted on the computing device responsive, at least in part, to an application of a dynamically determined rule set to the parsed parameters.

4. The method of claim 1, and further comprising:
storing, as signals and/or states in the memory device, a state of a patient graph comprising parameters expressing a profile of a medical patient; and
further executing the one or more processors to:
change signals and/or states in the memory device expressing the state of the patient graph responsive, at least in part, on the retrieved at least one of the event objects.

5. The method of claim 4, wherein the execution of the one or more applications hosted on the computing device is further based, at least in part, on the parameters expressing the profile of the medical patient.

6. The method of claim 4, wherein the signals and/or states in the memory device expressing the state of the patient graph are further changed based, at least in part, on parameters obtained from a data repository.

7. The method of claim 4, and further comprising executing the one or more processors to:
extract semantic features from the retrieved at least one of the event objects;
map the extracted semantic features to one or more vectors according to an encoding and/or embedding; and
change signals and/or states in the memory device expressing the state of the patient graph based, at least in part, on the one or more vectors.

8. The method of claim 4, wherein the parameters expressing the profile of the medical patient further comprises:
parameters expressing a health history of the medical patient;
parameters expressing demographics of the medical patient;
parameters expressing lab test results of the medical patient;
parameters expressing an observation or assessment of the medical patient; or
parameters expressing a health history of one or more family members of the medical patient;
parameters expressing financial status, insurance coverage, geography, housing status, employment status, education status;
parameters expressing a history of medical treatments received by the medical patient; or
parameters expressing a history of prescription drugs taken by the medical patient, or a combination thereof.

9. The method of claim 1, wherein:
the event objects are received at the computing device from one or more client computing devices over an electronic communication network, the received event objects having been created and formatted in the fields structured in the API at the one or more client computing devices.

10. The method of claim 1, and further comprising:
responsive to retrieval of the at least one of the event objects from the memory device, further executing the one or more processors to:
initiate execution of an instance of an autonomous trained agent to take action regarding initiation of clinical care, an administrative task and/or financial transaction.

11. The method of claim 10, wherein the action comprises a patient diagnostic procedure, a patient treatment procedure, a billing procedure and/or a compliance reporting procedure.

12. The method of claim 1, and further comprising further executing the one or more processors to:
extract one or more semantic attributes from the retrieved at least one of the event objects; and
changing a state of one or more compliance metrics and/or reports stored in a memory based, at least in part, on the extracted one or more semantic attributes.

13. The method of claim 1, wherein the transaction relates to an encounter of a medical patient with a medical service provider.

14. The method of claim 13, wherein the one or more parameters comprise a date and/or time of the transaction, identifier of an event type and identifier of a party to the transaction.

15. An apparatus comprising:
one or more communication devices to receive messages from a communication network;
a memory device to store, as signals and/or states, event objects in one or more messages received at the one or more communication devices, at least one of the event objects comprising one or more parameters to express a past event including a transaction to facilitate treatment, payment and/or healthcare operations; and
one or more processors to:
retrieve the at least one of the event objects from the memory device; and
initiate execution of one or more applications hosted on a computing device responsive, at least in part, to the retrieved at least one of the event objects based on an application programming interface (API), wherein:
each of the event objects comprising parameters that are expressible in fields structured in the API, the API being configured to initiate one or more of the applications hosted on the computing device responsive to event objects.

16. The apparatus of claim 15, wherein:
the memory device further stores, as signals and/or states, a state of a patient graph comprising parameters expressing a profile of a medical patient; and
the one or more processors are further to:
change signals and/or states in the memory device expressing the state of the patient graph responsive, at least in part, on the retrieved at least one of the event objects.

17. The apparatus of claim 16, wherein the execution of the one or more applications hosted on the computing device is further based, at least in part, on the parameters expressing the profile of the medical patient.

18. The apparatus of claim 16, wherein the signals and/or states in the memory device expressing the state of the patient graph are further changed based, at least in part, on parameters obtained from a data repository.

19. An article comprising:
a non-transitory storage medium having computer-readable instructions stored thereon which are executable by one or more processors of a computing device to:
store, as signals and/or states in a memory device, event objects obtained from one or more messages received from a communication network, at least one of the event objects comprising one or more parameters to express a past event including a transaction to facilitate treatment, payment and/or healthcare operations;
retrieve the at least one of the event objects from a memory device; and
initiate execution of one or more applications hosted on the computing device responsive, at least in part, to the retrieved at least one of the event objects based on an application programming interface (API), wherein:
each of the event objects comprising parameters that are expressible in fields structured in the API, the API being configured to initiate one or more of the applications hosted on the computing device responsive to event objects.

20. The article of claim 19, wherein:
the memory device further stores, as signals and/or states, a state of a patient graph comprising parameters expressing a profile of a medical patient; and
the instructions are further executable by the one or more processors to:
change signals and/or states in the memory device expressing the state of the patient graph responsive, at least in part, on the retrieved at least one of the event objects.
